(19) United States
(12) Patent Application Publication
(10) Pub. No.: US 2002/0123065 A1
(43) Pub. Date: Sep. 5, 2002

(54) Methods for making polynucleotide libraries, polynucleotide arrays, and cell libraries for high-throughput genomics analysis

(75) Inventors: Alan Lofquist, Kirkland, WA (US); Robert E. Finney, Shoreline, WA (US); David Leung, Mercer Island, WA (US)

Correspondence Address: Stephen A. Bent, Foley & Lardner, Washington Harbour, Suite 500, 3000 K Street, N.W., Washington, DC, 20007-5143, US

(73) Assignee: PanGenex, Inc.

(21) Appl. No.: 10/097,431

(22) Filed: Mar. 15, 2002

Related U.S. Application Data
(63) Continuation-in-part of application No. 10/028,970, filed on Dec. 28, 2001.
(60) Provisional application No. 60/258,388, filed on Dec. 28, 2000.

(51) Int. Cl. C12Q 1/68; G01N 33/53; C12M 1/34; C12N 15/74

(52) U.S. Cl. 435/6; 435/7.1; 435/320.1; 435/287.2

(57) ABSTRACT

A method for high-throughput genomics analysis, to identify the therapeutic or diagnostic utility of genes, entails the use of a construct to disrupt a gene or alleles of a gene in cells of interest. Arrays of such cells can be used to monitor such disrupted cells phenotypically in the context, for example, of testing drug candidates. Polynucleotides that comprise part of the disrupted genes can be recovered from such “knockout” cells, by virtue of an origin of replication or a host cell selection marker sequence that is part of the construct. The recovered polynucleotides can be used to identify the disrupted genes or to make homologous recombination vectors, which in turn can be employed to make multi-allele knockout cells.

This application is a continuation-in-part of U.S. patent application entitled, “Methods For Making Polynucleotide Libraries, Polynucleotide Arrays, And Cell Libraries For High-Throughput Genomics Analysis,” Ser. No. 10/028,970, filed Dec. 28, 2001, which claims priority to U.S. Ser. No. 60/258,388, filed Dec. 28, 2000, each of which is incorporated herein by reference. The present invention relates to novel cellular arrays, nucleotide trapping constructs and homologous recombination vectors that may be used to generate polynucleotide libraries, polynucleotide arrays and cell libraries, all of which can be useful in the context of high-throughput, functional-genomics analysis to decipher gene functions and to identify targets with therapeutic and/or diagnostic potential.

### FIELD OF THE INVENTION

### BACKGROUND OF THE INVENTION

The completion of various genome sequencing projects now provides the scientific community with a valuable resource of genetic information that serves as the foundation of gene-target discovery. However, deciphering and understanding the analyses of genomics-based assays can be difficult and ambiguous.

For instance, while there are numerous approaches for identifying genes, emerging technologies fail to provide a high-throughput means for identifying and using gene sequences. Among the large number of genes thus discovered, only a small fraction are likely to be, or to encode, valid gene-targets that have therapeutic or diagnostic utilities.

Many of these technologies correlate genes with human tissues, diseases and disorders. For example, “DNA array technology” is often used to correlate the expression pattern of a gene with specific tissues, diseases or disorders. Similarly, analyses of single nucleotide polymorphisms (SNPs) are used to detect mutations in DNA sequences and to correlate them with human diseases and disorders. Furthermore, proteomics can also be used to correlate expression of a protein with human tissues, diseases and disorders. Furthermore, proteomics is useful in determining interactions of a protein with other proteins thereby suggesting a role of the protein in a biochemical pathway. Direct examination of predicted structures of gene products identified in the human genome and comparisons to gene products with known functions (either other human genes or non-human organisms) can also be used to suggest biochemical properties or possible functions of a gene product.

Another approach has been to correlate gene expression with signaling pathways that have been implicated in cell phenotypes, including those associated with human diseases and disorders. In particular, gene trapping has been used to associate reporters with genes so that expression of genes in response to various environmental stimuli (such as growth factors) could be described. Whitney et al., Nature Biotechnol. 16: 1329-33 (1998); Medico et al., Nature Biotechnol. 19: 579-82 (2001). In these technologies, pools of cells containing vector DNA in non-prescribed locations were subjected to screening assays to identify cells that increased or decreased expression of the reporter in response to the stimuli. Identification of responding genes was then determined using conventional means. Although useful for some things, the utility of this technology has limitations. Using this technology, only genes actually responding to the stimulus are identified. Genes not responding are typically not identified. This makes cataloging of responding genes and non-responding genes difficult.

Recently, it has been shown that the combination of somatic cell genetics and fluorescence technology is useful in identifying agents that affect cellular processes thought to be critical to disease. Torrance et al., Nature Biotechnol. 19: 940-45 (2001). By co-culturing two, fluorescently-labelled, isogenic colon tumor cells lines, one of which contained an oncogenic K-ras allele, while the other had inactivated the oncogenic Kras allele. Torrance et al. was able to identify compounds that inhibited cell growth or cell survival, based upon relative intensities of fluorescent light emitted by protein markers introduced into those cells. However, the fluorescent protein markers were expressed constitutively by an exogenous regulatory system, not by an endogenous promoter. Accordingly, this method was not designed to identify gene targets, but rather, it was designed to identify agents differentially affecting growth or survival of cells lacking or containing the oncogenic Kras allele. Therefore, specific genes that may have served as potential diagnostic or drug discovery targets could not be determined.

While the aforementioned methods can be used to implicate gene products in human diseases and disorders, they do not directly demonstrate or correlate the role of gene products in the establishment or maintenance of such ailments. In particular, these methods fail to establish the phenotype of cells and tissues in which the function of the gene product is disrupted. Such correlative information is typically required to demonstrate the therapeutic utility of the gene product as a target for drug discovery.

Other technologies are used to gain direct information about effects of gene products on phenotypes associated with human tissues, diseases and disorders. Such information may be sought by: (i) over-expressing a gene product, (ii) disrupting a gene's transcript, such as by disrupting a gene's mRNA transcript; (iii) disrupting the function of a polypeptide encoded by a gene, or (iv) disrupting the gene itself.

Over-expression of a gene product, the use of antisense RNAs, ribozymes and double-stranded RNA interference (dsRNAi) techniques are also valuable in discovering inhibitors of gene products and for generating gene knockouts.

Over-expression of a target gene is often accomplished by cloning the gene or cDNA into an expression vector and introducing the vector into recipient cells. Alternatively, over-expression can be accomplished by introducing exogenous promoters into cells to drive expression of genes residing in the genome. The effect of over-expression on cell function, biochemical and physiology properties can then be evaluated. There are a number of disadvantages associated with this approach. For example, selecting cells that are suitable for over-expression of desired genes is not always straightforward. In addition, interpretation of the data from such experiments often is complicated by the fact that ectopically expressed genes are usually over-expressed at levels that are not physiologically relevant. Moreover, this approach does not shed light on the effect of under-expression of a gene, which may be critical to assessing the promise of the gene product as a drug target.

Antisense RNA, ribozyme, and dsRNAi technologies typically target RNA transcripts of genes, usually mRNA. Antisense RNA technology involves expressing in, or introducing into a cell, an RNA molecule (or RNA derivative) that is complementary to, or antisense to, sequences found in a particular mRNA into a cell. By associating with the mRNA, the antisense RNA can inhibit translation of the encoded gene product. Similarly, a ribozyme is an RNA that has both a catalytic domain and a sequence that is complementary to a particular mRNA. The ribozyme functions by associating with the mRNA (through the complementary domain of the ribozyme) and then cleaving (degrading) the message using the catalytic domain. Limited examples of use of double-stranded RNA (dsRNA) molecules, in a technique known as “RNA interference” are currently known for mammalian cells. It is believed that small (15-23 nucleotides, preferably 21-23 nucleotides) dsRNA molecules introduced into mammalian cells can associate with mRNA and induce degradation of that specific mRNA transcript (see WO 01/75164).

While such antisense, ribozyme and dsRNA methods have been used to evaluate functions of select genes, there are a number of disadvantages associated with these approaches. In particular, considerable time and effort is usually expended to identify reagents, such as dsRNA molecules, that inhibit gene product production to sufficient levels that a measurable or observable phenotype can be detected. That is, it can prove difficult to identify molecules that inhibit gene product production or activity by 30-50%, 60-80%, 80-90%, or 100% of their normal activity. In addition, non-specific effects are sometimes observed. Breakdown products for some of these molecules, also are known to elicit cellular responses such as induction of an interferon response. Therefore, lack of sufficient levels of inactivation and lack of specificity can lead to ambiguous interpretations as to the effect of any one of these approaches to gene inactivation or disruption. Consequently, considerable time and expense is expended by those seeking to generate and test such molecules that may directly or indirectly disrupt gene function in an efficient and precise manner.

In addition to using recombinant DNA technologies to disrupt gene function, chemical inhibitors also may be introduced into a cell to disrupt a gene or its protein product. However, to be useful, the biochemical function of a gene product is typically needed prior to implementation of the inhibitor. In this regard, it is useful to know of biological properties pertaining to the gene product prior to preparing such chemical assays. For instance, knowing the biochemistry of a protein (e.g. whether it has kinase or protease activity) can help to define the nature of the chemical assays to employ. With such information, cell-free, high-throughput screening assays can be usually established, a chemical diversity library obtained, and chemicals that inhibit the biochemical activity of a gene product selected. Cells in culture or animals can then treated with the chemical inhibitors to determine effects of an inhibitor on disease and disorder characteristics.

While such methods have been used to evaluate functions of select gene products, there are numerous disadvantages associated with these approaches. For example, the biochemical functions of most gene products encoded by the human genome are unknown or uncharacterized. In addition, establishing high-throughput assays for each gene product and screening for inhibitors demands significant resources and time for each potential target, which often means that only a few target genes can be evaluated at any one time. Most notably, inhibitors, especially early in compound discovery, are almost always non-specific for the gene product. Accordingly, the biochemical effect observed may not have been caused by inhibition of the targeted gene product. And finally, the method is further complicated by formulation problems and bio-availability of inhibitory compounds. This methodology is therefore costly and time consuming and the resulting information gathered is often non-definitive and ambiguous.

Perhaps the most unambiguous means to demonstrate the functions and therapeutic utilities of genes is by direct genetic disruption (including inactivation) by gene knockout technologies. The strategy in cell culture may involve the use of homologous recombination vectors to change (disrupt) a gene residing in a cell genome. For cultured cells, several rounds of homologous recombination are typically necessary to disrupt multiple copies (alleles), of endogenous genes. For animals, including mice, a single round of homologous recombination can be performed in totipotent cells, such as embryonic stem cells, which can then be used to generate a mouse that is heterozygous for the disrupted gene. Homozygous gene inactivation can then be accomplished by mating heterozygous animals. Gene disruption can also be accomplished using gene trapping technology to disrupt one copy of a gene in cell culture or a totipotent cell, such as an embryonic stem cell, and may be followed by identification of the disrupted gene and generation of homozygous mice.

The advantages of gene knockouts for determining the functions of genes are numerous. In particular, homologous recombination vectors offer complete inactivation of all alleles of a gene, which means unequivocal determination of gene function upon cell phenotype. Possible non-target associated effects, are usually minimal. Therefore, effects on cellular and animal phenotypes can be ascribed to a gene product with a very high degree of confidence. In addition, it is not necessary to know the biochemical function of the gene product before it is evaluated for function and therapeutic utility.

However, there are presently disadvantages with inactivating genes through the use of homologous recombination vectors. For example, conventional means for generating and using homologous recombination vectors to inactivate one or more genes in mammalian cells, including human cells, is labor intensive and costly. Typically, homologous recombination vectors are generated by cutting genomic DNA with specific endonucleases and cloning specific DNA fragments into vectors suitable for recombination. Alternatively, fragments are generated by polymerase chain reaction (PCR) and ligated into such vectors. For these reasons, gene inactivation in mammalian cells using homologous recombination has been limited and not amenable to high throughput.

In addition, although generation of mice with inactivated genes has been accomplished, analysis of functions, diagnostic and therapeutic utilities of these genes is hindered by the observation that many gene disruptions cause embryonic lethality. Characterization of gene function in adult animals therefore requires many additional methods, which can be expensive and laborious. Additional utility of mice is hindered by lack of relevant disease models for human diseases. And most notably, mice are also not typically used for high throughput assays.

In sum, there are significant drawbacks in conventional methods of evaluating the therapeutic and diagnostic potential of genes and gene products. Such methods tend to be resource-intensive, costly and, in many cases, interpretation of the results is ambiguous. Moreover, they are marked by relatively low throughput and, hence, are hard-pressed to meet the challenge of high-throughput analysis of gene product function, as well as diagnostic and therapeutic utility.

### SUMMARY OF THE INVENTION

One need that the present invention addresses is for an approach to high-throughput analysis of genes and gene products, to identify therapeutic or diagnostic utilities for these genes and gene products with high confidence. Another need that the invention addresses relates to an improved methodology for identifying genes.

Thus, the instant invention provides an array of cells, organized in a systematic fashion that can be used for a number of purposes. The array may contain cells of a certain type or cells in which the expression of multiple alleles of a gene has been disrupted. Accordingly, the array lends itself to the simultaneous analysis of, for example, the phenotypes of the arranged cells, or the effects of a drug or compound upon the arranged cells.

In another embodiment, an array of cells is provided that comprises multiple groups of vessels, of which at least two of vessels contain cells, wherein each vessel of cells has the expression of at least one gene or allele of a gene disrupted. In a further embodiment, the vessels containing the disrupted gene or allele is arranged in the array in a predetermined fashion. In yet another embodiment, the cells are placed into vessels of an array in some predetermined fashion. In a preferred embodiment, at least some of the cells differ from other cells by virtue of the gene or allele expression disrupted.

Accordingly, the instant invention provides an array of clones, comprising multiple groups of vessels, of which at least two of the vessels each contain a clone of a cell, wherein each clone (i) contains an exogenous segment within a gene of its genome, such that the gene is disrupted, and (ii) is arranged in predetermined fashion.

The instant invention provides an array of cells, comprising multiple groups of vessels, of which at least two of the vessels each contain cells, wherein each vessel of cells (i) contains a gene whose expression is inhibited or altered at the transcriptional level and (ii) is arranged in predetermined fashion in the array.

The instant invention provides an array of cells, comprising multiple groups of vessels, of which at least two of the vessels each contain cells, wherein each vessel of cells (i) contains a gene whose expression is inhibited or altered at the translational level and (ii) is arranged in predetermined fashion in the array.

The instant invention provides an array of cells, comprising multiple groups of vessels, of which at least two of the vessels each contain cells, wherein each vessel of cells (i) contains a gene whose expression is inhibited or altered at the protein level and (ii) is arranged in predetermined fashion in the array.

Furthermore, the instant invention provides an array of vessels, wherein each vessel contains more than one clone, wherein each clone contains a different disrupted gene. In one embodiment, each clone contains an exogenous segment within a gene of its genome, such that the gene is disrupted. In a further embodiment, the vessels are arranged in a predetermined fashion in the array.

Also provided by the instant invention is an array of vessels, each of which contains more than one clone. Each clone contained in a vessel comprises at least one gene allele that cannot express a functional gene product. In another embodiment, each clone contained in a vessel comprises at least one gene allele that is expressed at a lower than normal level. In yet another embodiment, each clone contained in a vessel is different from other clones also contained in the same vessel by virtue of its dysfunctional gene allele. In one embodiment the expression of a gene allele of a clone in a vessel is inhibited by an exogenous molecule. In another embodiment, the expression of a gene allele may be inhibited by an antisense nucleotide, a double-stranded RNA molecule, a ribozyme, or a chemical inhibitor. In yet another embodiment the vessels of the array are all arranged in a predetermined fashion.

Yet another aspect of the instant invention envisions an array of cells, wherein each cell comprises at least one gene allele targeted by an exogenous molecule that reduces or abolishes the expression of its normal protein product. In one embodiment, the expression of a gene allele may be inhibited by an antisense nucleotide, a double-stranded RNA molecule, a ribozyme or a chemical inhibitor. In a further embodiment, the cells of the array are arranged in a predetermined fashion.

In a preferred embodiment, the exogenous segment is a construct or a portion thereof, that comprises an origin of replication or a cell selection marker, preferably a host cell selection marker.

In another preferred embodiment, at least some of the clones in an array differ by virtue of the gene within which the exogenous segment is integrated.

In yet another preferred embodiment, each of the clones in an array differs from every other clone of the array by virtue of the gene of its genome within which the exogenous segment is integrated.

In a preferred embodiment, some, or each, of the clones in an array contains the same disrupted gene.

In another aspect of the invention, each gene is an allele and the genome in which the gene is found, contains one or multiple alleles of that particular gene.

Accordingly, the array of clones may comprise at least one allele disrupted by an exogenous segment. In a preferred embodiment, every one of the multiple alleles is disrupted an exogenous segment. In a preferred embodiment, the array may contain as few as 2 cells. Preferably any array of the instant invention contains between 2-10 cells, 10-20 cells, 20-40 cells, 40-60 cells, 60-80 cells, 80-100 cells, 100-200 cells, 200-300 cells, and more than 300 cells.

In yet another aspect of the invention, an array of clones is provided that comprise multiple groups of vessels, of which at least two of vessels each contain a clone, wherein each clone contains (i) an first exogenous segment within a first gene of its genome, such that the first gene is disrupted, and (ii) a second exogenous segment within a second gene of its genome, such that the second gene is disrupted. Accordingly, this multigene-knockout cell may be useful in establishing the molecular relationship, if any, between at least two different genes and their respective gene products, in a cell.

The instant invention also provides a construct comprising certain functional elements that, when transcribed, produce a eukaryote mRNA transcript (i.e., one that can be translated in a eukaryote cell) containing one or both of an origin of replication and a host cell selection marker. The construct preferably is integrated into a genome of a cell, especially in a transcribable region and, even more preferably, into an endogenous gene. Thus, transcription of that endogenous gene would result in the production of an mRNA transcript that contains at least one functional element of the construct, such as an origin of replication or a host cell selection marker.

In one embodiment, the construct also contains (i) a splice acceptor sequence; and at least one of a (ii) an origin of replication and (iii) a host cell selection marker. In a preferred embodiment, the splice acceptor sequence is upstream to the origin of replication or to the host cell selection marker.

In another preferred embodiment, the construct further comprises a means for facilitating termination and polyadenylation of an endogenous polynucleotide that is downstream from the origin of replication or the host cell selection marker. Accordingly, the means for facilitating termination and polyadenylation of an endogenous polynucleotide can be a termination sequence, such as a polyadenylation sequence. In a preferred embodiment, the means for facilitating termination and polyadenylation of an endogenous polynucleotide is a splice donor sequence. In another preferred embodiment, the construct may contain (i) an IRES sequence; and at least one of a (ii) an origin of replication and (iii) a host cell selection marker.

In another embodiment, the cell in which the construct is integrated is a eukaryotic cell and can be a non-human cell or a human cell. Preferably, the cell is a somatic cell or a germ cell. In a preferred embodiment, the germ cell is a stem cell. The cell in which the construct, or a portion thereof, is integrated also may be a non-dividing cell or one proliferate in vitro.

In yet another preferred embodiment, the cell in which the construct is integrated has disease attributes, selected from the group consisting of, but not limited to, a tumor cell, a colon cancer cell or a Kras-transformed colon cancer cell.

The present invention also provides a construct comprising (A) a splice acceptor site, (B) a cassette sequence selected from the group consisting of (i) a transcriptional termination sequence and (ii) a splice donor site, (C) a cell selection marker sequence, and (D) an origin of replication, wherein the origin of replication and the marker sequence are located downstream to the 5′-end of the splice acceptor site and upstream to the 3′-end of the cassette sequence, and wherein the origin of replication is exogenous to the splice acceptor site or the cassette sequence. In a preferred embodiment, the construct comprises an IRES and a Shine-Dalgarno sequence, both of which are located downstream to the 3′-end of the splice acceptor site and upstream to the 5′-end of the open-reading frame. In a preferred embodiment, the cell selection marker is a host cell selection marker.

According to another aspect of the invention, a construct has been provided that comprises (A) a transcriptional initiation sequence, (B) a splice donor site, (C) a cell selection marker sequence, and (D) an origin of replication, wherein the origin of replication and the cell selection marker sequence are located downstream to the 5′-end of the transcriptional initiation sequence and upstream to the 3′-end of the splice donor site, and wherein the origin of replication is exogenous to the transcriptional initiation sequence or to the splice donor site.

The invention further encompasses, according to another aspect, a method for obtaining a polynucleotide that comprises (A) providing a target cell in which at least one allele of a gene in the genome of the target cell is disrupted by a construct, wherein the construct comprises an origin of replication, and wherein the origin of replication is exogenous to the target cell and is capable of initiating DNA synthesis in a host cell, and (B) selecting a polynucleotide from the target cell, wherein the polynucleotide comprises the origin of replication and a genomic sequence comprised in the gene. The genomic sequence can then be identified according to conventional techniques and the particular region of the genome disrupted identified.

In a preferred embodiment, the construct comprises a target cell selection marker sequence encoding a target cell selection marker that confers a selectable trait to the target cell. In another preferred embodiment the construct comprises a prokaryotic selection marker sequence encoding a prokaryotic selection marker that confers a selectable trait to the prokaryotic cell.

In yet another preferred embodiment, the construct further comprises a splice acceptor site and a cassette sequence selected from the group consisting of (i) a transcriptional termination sequence and (ii) a splice donor site, wherein the origin of replication, the target cell selection marker sequence and the prokaryotic selection marker sequence, if present, are located downstream to the 5′-end of the splice acceptor site and upstream to the 3′-end of a cassette sequence. In yet another preferred embodiment, a method of making a polynucleotide array is provided which comprises spotting onto a suitable array medium a polynucleotide, or fragment thereof.

According to another aspect of the invention, a linear vector has been provided that comprises, in the 5′ to 3′ order, (A) a first nucleotide sequence that is homologous to a first genomic sequence of a cell, (B) a construct comprising an origin of replication that is exogenous to the cell, and (C) a second nucleotide sequence that is homologous to a second genomic sequence of the cell. In a preferred embodiment, the vector provided comprises a negative selection marker sequence located either 5′ to the first nucleotide sequence or 3′ to the second nucleotide sequence.

In another aspect of the invention, a vector library has been provided that comprises at least two vectors, wherein each vector comprises (A) a first nucleotide sequence homologous to a first genomic sequence of a cell, (B) a construct comprising a marker sequence, and (C) a second nucleotide sequence homologous to a second genomic sequence of the cell, and wherein the gene represented by any given vector is different from the gene represented by any other vector in the library.

In a further aspect of the instant invention, a homologous recombination vector is provided. The homologous recombination vector comprises, (i) a splice acceptor sequence or an IRES sequence; (ii) an origin of replication or a host cell selection marker; (iii) a means for facilitating termination and polyadenylation of an endogenous polynucleotide; (iv) a first genomic fragment recovered from a cell; and (v) a second genomic fragment recovered from a cell, wherein (ii) is downstream of (i) and upstream of (iii), and wherein the first genomic fragment is upstream of (i) and the second genomic fragment is downstream of (iii) and wherein the first and second genomic fragments are capable of undergoing a homologous recombination event.

A method for making a homologous recombination vector also is provided in the instant invention. In one embodiment, the method comprises, (i) integrating a construct into the genome of a cell; (ii) recovering a polynucleotide comprising at least a portion of the construct that is flanked at either its 5′-end or its 3′-end, or at both of its ends by a genomic fragment of the genome; and (iii) isolating the polynucleotide to form a homologous recombination vector. Preferably, the construct contains an origin of replication or a host cell selection marker. The flanking genomic fragments can be sequenced and the gene identified. Accordingly, by comparing the identified gene sequence to that of a published genome, one may be able to determine the location at which the exogenous DNA or construct integrated within the genome, as well as flanking genomic DNA sequences.

It is also an aspect of the instant invention to isolate mRNA transcripts from transcribed regions of the genome that contain an integrated construct or a portion thereof, that is, an exogenous segment. The mRNA transcript, therefore, will contain, within its ends, the RNA of the exogenous segment. Preferably, the exogenous segment contains an origin of replication. Accordingly, a cDNA of the mRNA can be made according to standard techniques and used as a template from which to sequence the polynucleotides that flank the origin of replication. In this way, it is possible to identify the exonic regions of a transcribed region of the genome. That is, in this fashion it is possible to directly identify a coding region of a gene.

In a preferred embodiment, once a construct has been integrated into a genome, the genome may be fragmented to produce polynucleotides of which, at least one fragment will contain a portion of the integrated construct. In a preferred embodiment, the fragmenting of the genome may be accomplished by restriction enzyme digestion or by mechanical forces.

In another preferred embodiment, a construct may be integrated into a genomic preparation of DNA that is extracted and purified from a cell. Accordingly, in one aspect of the instant invention, a construct may be integrated into a purified nucleic acid preparation in vitro. In a preferred embodiment, therefore, a homologous recombination vector may be prepared by integrating a construct into genomic DNA in vitro, instead of in a cell in vivo.

In another aspect of the instant invention, a positive switch homologous recombination vector is provided which comprises (i) a splice acceptor sequence or an IRES sequence; (ii) a first termination sequence; (iii) a positive selection marker; (iv) a first genomic fragment recovered from a cell; and (v) a second genomic fragment recovered from a cell. In a preferred embodiment, these elements are arranged such that (ii) is upstream of (i), (iii) and (iv), and (v) is downstream of (i), (iii) and (iv). In a preferred embodiment, the positive switch homologous recombination vector may further comprise a second termination sequence located downstream of the first termination sequence. In a preferred embodiment, both first and second termination sequences may be polyadenylation sequences. In a preferred embodiment, the second polyadenylation sequence is 3′ to the positive cell selection marker. In another preferred embodiment, both first and second termination sequences may be splice donor sequences. In yet another embodiment, the first termination sequence may be either a polyadenylation sequence or a splice donor sequence. Accordingly, in another embodiment, the second termination sequence may be either a polyadenylation sequence or a splice donor sequence.

According to another aspect of the invention a cell, comprising (i) an allele of a first gene into which an exogenous polynucleotide has been integrated, is provided. In a preferred embodiment, the exogenous polynucleotide contains an origin of replication or a selectable marker upstream of a means for facilitating termination and polyadenylation of an endogenous polynucleotide and downstream from a transcription initiation sequence. In a preferred embodiment, the means for facilitating termination and polyadenylation of an endogenous polynucleotide include splice donor sequences or termination sequences such as a polyadenylation sequence.

Still another aspect of the invention relates to a cell wherein at least one allele of a gene in the genome of the cell is disrupted by a construct, which comprises an origin of replication exogenous to the cell. In a preferred embodiment, a cell library is provided that comprises at least two cells, wherein the gene disrupted in any given cell is different from genes disrupted in other cells in the library.

In an additional aspect of the invention, a cell has been provided wherein each allele of a gene in the genome of the cell is disrupted by a construct comprising an origin of replication exogenous to the cell.

According to another aspect of the invention the making of a cell in which at least two alleles of a gene are disrupted by a construct exogenous to the cell is provided, where the cell is produced by (1) disrupting a first allele of the gene, via a method other than homologous recombination, by a construct exogenous to the cell; and (2) disrupting a second allele of the gene using a homologous recombination vector exogenous to the cell, wherein the vector comprises a first nucleotide sequence homologous to a first genomic sequence of the cell, (b) a cell selection marker sequence, and (c) a second nucleotide sequence homologous to a second genomic sequence of the cell, and wherein the gene comprises the first and second genomic sequences. In a preferred embodiment, the first and second genomic sequences share sequence homology with the second allele of the gene, such that homologous recombination can occur between the vector and the second allele.

Preferably, the genomic sequences are of sufficient length so as to be capable of undergoing a homologous recombination event. In a preferred embodiment, each genomic fragment is at least 50 nucleotides in length.

In yet another aspect, a cell is provided that contains more than one gene disrupted by a construct or by a homologous recombination vector or by both. That is, a cell may contain a first gene and a second gene that are disrupted by the integration of a construct of the instant invention. In a preferred embodiment, each allele of both the first gene and the second gene are disrupted by the integration of a construct into the allelic nucleotide sequences.

Accordingly, the instant invention provides cells with a single allele disrupted by a construct; cells with two alleles disrupted by a construct; and cells with all alleles disrupted by a construct or a portion thereof. In a preferred embodiment, the construct may be a construct or a homologous recombination vector, or any construct that is capable of integrating into genomic DNA. In another preferred embodiment, any construct that is used to integrate exogenous nucleotides into a genome of a cell or into a preparation of DNA may further comprise a sequence encoding a reporter marker. When expressed, the reporter marker sequence produces a polypeptide whose activity or presence indicates the extent to which the endogenous regulatory elements associated with the gene in which a construct is integrated, is active.

The present invention envisions the creation of libraries of each of these particular cells. Accordingly, yet another aspect of the invention provides a cell library that comprises at least 2 cells, wherein at least one allele of a gene in the genome of each cell is disrupted by a construct exogenous to the cell, and wherein the gene disrupted in any given cell is different from the gene disrupted in any other cell in the library.

In a further aspect of the invention, there has been provided a cell library that comprises at least two cells, wherein the genome of each cell comprises a gene, each allele of which is disrupted by a construct exogenous to the cell, and wherein the gene disrupted in any given cell is different from the gene disrupted in any other cell in the library.

A library of constructs wherein each construct produces, upon transcription, an mRNA transcript containing an exogenous origin of replication or a host cell selection marker, is also provided. In a preferred embodiment, at least some of the mRNA transcripts produced by the constructs represent different gene sequences. In another preferred embodiment, each of the mRNA transcripts produced by the constructs represents a region of a cell genome that does not encode a polypeptide.

In yet another aspect of the instant invention, a method for recovering at least a portion of a gene allele of a cell genome is provided. This method entails (i) providing a cell in which at least one allele of a gene in the genome of the cell contains a construct, or a portion thereof, within any part of its nucleotide sequence, wherein the construct or a portion thereof, comprises an origin of replication or a host cell selection marker; (ii) recovering a nucleic acid molecule containing the construct, or a portion thereof, that contains an origin of replication or a host cell selection marker and nucleic acid derived from the allele; and (iii) isolating the recovered nucleic acid molecule. In a preferred embodiment, the recovered and isolated nucleic acid derived from the allele flanks either the 5′-end, the 3-′ end or both ends of the construct, or a portion thereof.

Accordingly, the resultant nucleic acid contains a construct, or a portion thereof, flanked by a fragment of the allele into which it was integrated. In a preferred embodiment, only one end of the construct, or portion thereof, is flanked by an allelic fragment. In another preferred embodiment, both ends of the construct, or portion thereof, are flanked by an allelic fragment. In a preferred embodiment, the allelic fragments can be isolated and/or sequenced to identify their identity. Alternatively, the resultant nucleic acid may be used directly as a homologous recombination vector.

According to another aspect of the invention, a method for determining the function of a gene has been provided that comprises (i) providing a first cell, wherein at least two alleles of the gene in the genome of the cell are disrupted by a construct exogenous to the cell; and (ii) comparing biological traits of the first cell to those of a second cell in which no alleles of that particular gene have been disrupted.

In another aspect of the invention, a method for selecting a drug candidate that regulates expression of a reporter marker integrated into at least one allele of a gene in a cell. In a preferred embodiment, the reporter marker is a fluorescent protein. A preferable method comprises, (i) contacting a drug candidate with a cell wherein at least one allele of a gene in the genome of the cell is disrupted; and (ii) comparing the fluorescent light intensity of the reporter marker sequence in the cell before and after contacting the drug candidate.

In yet one other aspect of the present invention, a method of interfering with the operation of a target gene in a cell is provided. For example, compositions that target the inhibition of expression or activity of a gene product are envisioned. Examples of such compositions include, but are not limited to, antisense sequences, ribozymes and chemical inhibitors. The “chemical inhibitor” category includes but is not limited to chemical protein inhibitors.

In one embodiment, a double-stranded RNA molecule is introduced into a cell that targets the mRNA of a gene based upon its homology to the sequence of the predicted mRNA transcript of the gene. Preferably, the association of the dsRNA is defined by a homology to the actual and/or predicted mRNA transcript that is 50-100%, 60-100%, 70-100%, 80-100%, 80-90%, 80-95%, 90-100% or 95-100%. In this description, “double-stranded RNA” or “dsRNA” denotes RNA that has the characteristics described above. The RNA molecule may be double-stranded or single-stranded RNA that can anneal to itself to form a hairpin structure. The RNA also may be “isolated” RNA; that is, the RNA may be partially purified RNA, essentially pure RNA, synthetic RNA, or recombinantly produced RNA. The RNA may be altered and may differ from naturally occurring RNA by the addition, deletion, substitution and/or alteration or one or more nucleotides. Such alterations may also include addition of non-nucleotide material to the ends of the dsRNA. Alternatively, modifications can be made to the ends and within the RNA molecule, including the addition of, non-standard nucleotides or deoxyribonucleotides.

In one embodiment, the dsRNA molecule is introduced into a cell by any of the methods selected from the group comprising, but not limited to, electroporation, transfection and lipofection.

In another embodiment, a double stranded RNA molecule may comprise individual nucleic acid strands that are of equal length. In a preferred embodiment, each strand of the dsRNA molecule is about 5-100 bp, 5-50 bp, 5-45 bp, 5-40 bp, 5-35 bp, 5-30 bp, 5-25 bp, 5-20 bp, 5-15 bp, 5-10 bp, 10-15 bp, 10-20 bp, 10-25 bp, 20-25 bp, 21-23 bp, 10-30 bp, 15-25 bp, 15-30 bp or 15-20 bp in length.

In another aspect of the instant invention, a library of dsRNA molecules is provided, wherein each dsRNA molecule in the library is associated with a target gene or target genes. In a preferred embodiment, each of the dsRNA molecules is capable of inhibiting the expression of a gene product. In one embodiment, the dsRNA library comprises anywhere from at least 2 to 10, 10-20, 20-30, 30-40, 40-50, 50-100, 100-200 or more than 200 dsRNA molecules.

Another aspect of the present invention provides for one or more cells wherein the operation of a target gene is disrupted by introduction of a dsRNA molecule that is associated with the target gene. In a preferred embodiment, the cell and/or cells of the library may be prokaryotic, eukaryotic or viral. In another embodiment, a library of cells modified by dsRNA molecules is envisioned, wherein each cell of the library comprises one or more a dsRNA-modulated target genes. In one embodiment, the dsRNA-cell library comprises from at least 2 to 10, 10-20, 20-30, 30-40, 40-50, 50-100, 100-200 or more than 200 cells.

In another aspect, a method of modulating the expression of a target gene in a cell is provided, comprising introducing into a cell a dsRNA molecule that shares sequence homology with the actual and/or predicted mRNA transcript of a target gene. The dsRNA molecule may share sequence homology and be complementary to a part of a target gene, preferably to a region of approximately 20-30 nucleotides

In a further aspect of the instant invention, an mRNA transcript is provided and encoded by a construct that comprises in 5′ to 3′ order, a promoter, a polynucleotide of interest, an IRES sequence and a reporter polynucleotide that are operably linked to one another. In another embodiment, an mRNA transcript is provided by another construct that comprises, in 5′ to 3′ order, a promoter, a reporter polynucleotide, an IRES sequence and a polynucleotide of interest that are operably linked to one another. A dsRNA molecule can be used to target such mRNA transcript based upon its homology. A polynucleotide of interest need not encode an entire gene, but may encode any DNA sequence for which an associated dsRNA can be produced.

The instant invention also provides a method for determining the effectiveness of a dsRNA molecule comprising: (a) introducing a construct into a cell; and (b) detecting the activity or presence of a reporter molecule encoded by the construct after exposing the cell to dsRNA. In one embodiment, the cell into which the dsRNA molecule is introduced may contain a single-allele gene disruption. Alternatively, an antisense sequence, a ribozyme, or chemical inhibitor, for example, may be introduced into such a cell and the activity or presence of a reporter molecule encoded by the construct similarly detected.

Any method of introducing or producing dsRNA into a cell as known in the art is contemplated, including, but not limited to, introducing synthetic dsRNA and introducing materials into the cell to produce dsRNA.

A method for determining the effectiveness of a modulator of gene expression in vitro or in vivo may be performed on genes that are over-expressed by recombinant methods. Accordingly, the effectiveness of a molecule to modulate gene expression, wherein the expression is not normal, may not represent the most appropriate conditions for determining the effectiveness of such a modulator in vivo. Accordingly, a cell into which a trap construct that contains a reporter marker has been integrated into its genome, may be subjected to a molecule, such as a dsRNA, an antisense polynucleotide, ribozyme or chemical inhibitor that targets the gene into which the trap has been integrated. The effect of the molecule on the expression of the reporter marker therefore is an indicator of the effect of the molecule in inhibiting expression of that particular gene. Thus, the in vivo method for determining inhibition effectiveness can be compared to the over-expressed in vitro method, and an optimized set of conditions for inhibiting or reducing gene expression can be determined.

A method for determining the effectiveness of a double-stranded RNA molecule also is provided. In a preferred embodiment, the method comprises (i) introducing into a cell with one allele disrupted by an exogenous polynucleotide, a construct comprising: (a) a promoter; (b) a polynucleotide of interest; (c) an IRES sequence; and (d) a reporter marker; (ii) determining the activity or expression level of the reporter marker; (iii) introducing into the cell a double-stranded RNA molecule designed to a portion of the polynucleotide of interest; and (iv) determining the activity or presence of the reporter marker.

Accordingly, as it applies to the use of doublestranded RNA to modulate expression of a gene, it is preferable that such a construct does not disrupt an allele's endogenous regulatory elements that drive expression of the allele. Thus, it is preferable that the construct does not disrupt a promoter associated with the allele into which a construct, or a portion thereof, is integrated.

In yet another aspect, a method of up-regulating the expression of a target gene in a cell, comprises introducing into a cell a dsRNA molecule that is capable of associating with a second gene that normally inhibits or reduces the expression of the target gene.

The instant invention also comprehends the automation of many of the molecular biology procedures described herein. These automations effect the inventive methodology in such a way that robotic instruments routinely perform, for example: (1) “feeding” of colonies (2) selecting colonies and pooling them; (3) splitting cells into cultures and freezing; (4) performing genomic DNA and RNA preps and subsequent RT-PCR, cDNA steps; and/or (5) assays of cells.

Other features, objects, and advantages of the present invention are apparent in the detailed description that follows. It should be understood, however, that the detailed description, while indicating preferred embodiments of the invention, are given by way of illustration only, not limitation. Various changes and modifications within the spirit and scope of the invention will become apparent to those skilled in the art from the detailed description.

### DETAILED DESCRIPTION OF THE PREFERRED EMBODIMENTS

The present invention imparts the capability to produces a cell that contains one or more inactivated gene alleles. In addition, polynucleotide fragments of such a disrupted gene allele can be isolated and sequenced, pursuant to the invention, thereby to illuminate the identity of the gene.

Such polynucleotides or fragments thereof can be used to create homologous recombination vectors, to target and disrupt remaining alleles of the same gene in a cell. Thus, the invention provides an efficient and precise way to produce a “knockout” cell that is unable to produce a transcript or to express a gene product of a gene or multiple alleles of a gene. Moreover, one readily can correlate the identity of a knockout cell with a corresponding polynucleotide and recombination vector, respectively.

The instant invention also provides arrays of cells, arranged in a predetermined fashion, that enables the simultaneous analysis of different cell types, phenotypes and genetic modifications. A particular embodiment of the invention is an array of multiple-allele knockout cells.

This description employs terms and phrases that are well known to the fields of molecular biology and genomics. Unless defined otherwise, all technical and scientific terms used here in a manner that conforms to common technical usage. Generally, the nomenclature of this description and the described laboratory procedures, in cell culture, molecular genetics, and nucleic acid chemistry and hybridization, respectively, are well known and commonly employed in the art. Standard techniques are used for recombinant nucleic acid methods, polynucleotide synthesis, microbial culture, cell culture, tissue culture, transformation, transfection, transduction, analytical chemistry, organic synthetic chemistry, chemical syntheses, chemical analysis, and pharmaceutical formulation and delivery. Generally, enzymatic reactions and purification and/or isolation steps are performed according to the manufacturers' specifications. Absent an indication to the contrary, the techniques and procedures in question are performed according to conventional methodology disclosed, for example, in Sambrook et al., Molecular Cloning A Laboratory Manual, 2d ed., Cold Spring Harbor Laboratory Press, Cold Spring Harbor, N.Y. (1989), and Current Protocols in Molecular Biology, John Wiley & Sons, Baltimore, Md. (1989).

Allele: An “allele” is a single copy of a gene and may be one of a pair or of a series of copies or variant forms of a gene.

Allelic: The term “allelic” connotes the existence of more than one copy or form of a particular gene. Thus, a gene is said to be allelic if it has more than one allele.

Array: In the present description, an “array” is an integral collection of objects that may be arranged in a systematic manner or in some predetermined fashion. An “array” can be, for example, an integral collection of vessels or an integral collection of wells. That is, an “array” can be a collection of objects that are formed as a unit with another part. An “array” also can be a surface upon which an integral collection of substances are arranged in a systematic manner.

Array of cells: An “array of cells” is a collection of cells, arranged in a systematic manner. An “array of cells,” or a “cell array,” represents, for example, a non-random arrangement of cell types or cells in which a gene is disrupted, contained within an integral collection of vessels or wells.

Cell: A “cell” of the instant invention may be, but is not limited to, a host cell, a target cell, a healthy cell, a mutated cell, a cell with disease or disorder characteristics (“diseased cell”), a transformed cell or a modified cell. A “cell” in this description may also denote a culture of such cells. A modified cell may be a cell that contains within its genome an integrated “construct” or an integrated “exogenous segment.” Such a cell may be regarded as a “knockout.” A modified cell may contain a polynucleotide whose expression is regulated by a biological factor or groups of such factors. In this respect, a modified cell may be a cell that contains a regulatable gene.

Clone: A “clone” is a number of cells with identical genomes, derived from a single ancestral cell. Thus, a group of genetically identical cells produced by mitotic divisions from one original cell, are “clones.” According to the instant invention, a clone represents at least one cultured, preferably non-frozen cell, or plurality of such cells, each tracing its lineage to one cell.

Construct: The term “construct” denotes an artificially assembled polynucleotide molecule, such as a cloning vector or plasmid, that can exist in linear or circular forms. Typically, a construct will include elements such as a gene, a gene fragment, or a polynucleotide sequence of particular interest, juxtaposed with other elements in the construct, such as a cell selection marker, a reporter marker, an appropriate control sequence, a promoter, a termination sequence, a splice acceptor site, a splice donor site, and restriction endonuclease recognition sequences (multiple cloning sites). A construct may be, for example, a “trap construct” or a homologous recombination vector. A construct, or a part of it, may be integrated into a genome of a cell or into an in vitro-prepared preparation of a cell genome. Thus, an “integrated construct” can mean that an entire construct has been inserted into a genome or it can mean that a portion of a construct has been integrated into the genome. The latter may contain functional elements that are present in the intact construct, such as an origin of replication or a host cell selection marker. Accordingly, a portion of a construct may constitute an “exogenous segment.”

Disrupted: “Disrupted” means the hindering of the expression of an endogenous gene product. In one embodiment, an allele of a gene is “disrupted” if any part of the allele nucleotide sequence contains a construct. Thus, a nucleotide sequence naturally present in a cell genome can be “disrupted” by the integration of another nucleotide sequence between a 5′ end and a 3′ end of the former sequence. The nucleotide sequence that disrupts a gene in a cell genome may be flanked by regions that, but for the presence of the sequence, together encode a polypeptide. Disruption of a gene by a construct, for example, may result in non-expression of a gene product in a cell or in the expression of a partially or totally non-functional gene product or an altered gene product.

dsRNA: A “dsRNA” or “double-stranded RNA” molecule refers to RNA having the characteristics described above. The RNA molecule may be double-stranded, or single-stranded RNA that can anneal to itself to form a hairpin structure. The RNA also may be isolated RNA, that is, the RNA may be partially purified RNA, essentially pure RNA, synthetic RNA, or recombinantly produced RNA. The RNA may be altered and may differ from naturally occurring RNA by the addition, deletion, substitution and/or alteration or one or more nucleotides. Such alterations may also include addition of non-nucleotide material to the ends of the dsRNA. Alternatively, modifications can be made to the ends and within the RNA molecule, including the addition of, non-standard nucleotides or deoxyribonucleotides.

Downstream: A polynucleotide sequence in a construct is regarded as being downstream or 3′ to a second polynucleotide sequence in the construct, if the 5′ end of the former sequence is located after the 3′ end of the latter sequence.

dsRNA-modulated gene: A “dsRNA-modulated gene” refers to a gene whose expression product has been inhibited by a dsRNA molecule

Exogenous: A nucleotide sequence is “exogenous” to a cell if it is not naturally a part of that cell genome, or it is deliberately inserted into the genome of the cell. A nucleotide sequence may be deliberately inserted into a cell genome by human intervention or automated means.

Exogenous segment: An exogenous nucleotide sequence, such as the sequence of a construct, or a portion thereof, may be referred to as an “exogenous segment.” An exogenous segment may contain functional elements present in the intact construct, such as an origin of replication or a host cell selection marker.

Gene: A “gene” contains not only the exons and introns of the gene but also other non-coding and regulatory sequences, such as enhancers, promoters and the transcriptional termination sequence (e.g. the polyadenylation sequence). As used in this description, a gene does not include any construct that is inserted therein by human intervention or by automation. A gene may be allelic in nature.

Genome: The “genome” of a cell includes the total DNA content in the chromosomes of the cell, including the DNA content in other organelles of the cell, such as mitochondria or, for a plant cell, chloroplasts.

Genomic sequence: A “genomic sequence” of a cell refers to the nucleotide sequence of a genomic DNA fragment of the cell.

Host cell: Suitable host cells may be non-mammalian eukaryotic cells, such as yeast, or preferably, prokaryotic cells, such as bacteria. For instance, the host cell may be a strain of E. coli.

Homologous recombination: The term “homologous recombination” refers to the process of DNA recombination based on sequence homology of nucleic acid sequences in a construct with those of a target sequence, such as a target allele, in a genome or DNA preparation. Accordingly, the nucleic acid sequences present in the construct are identical or highly homologous, that is, they are more than 60%, preferably more than 70%, highly preferably more than 80%, and most preferably more than 90% sequence identity to a target sequence located within a cell genome. In a particular embodiment, a the homologous recombination vector has 95%-98% sequence identity to a target sequence located within a cell genome.

Integral: The word “integral” means formed as a unit with another part. Accordingly, applying the characterization of “integral” to a collection of elements, such as of wells or of vessels, indicates a purposeful accumulation of interrelated elements that are arranged in some predetermined fashion. An “integral” plurality of elements may refer to some but not necessarily to all elements of an array, for example. “Integral” also may be used to describe the contents within wells or vessels of an inventive array.

Isolated polynucleotide: “Isolated” means to separate from another substance so as to obtain pure or in a free state. Accordingly, an “isolated polynucleotide,” is a polynucleotide that has been separated from other nucleic acids, such as from a genome of a cell or from a genomic DNA preparation, or from other cellular compositions.

Knockout: “Knockout” means having a specific single gene or allele(s) of a gene disrupted from a genome by genetic manipulation. Accordingly, a “single-allele, knockout cell” refers to a cell in which a single allele of a gene has been disrupted such that its gene product is not expressed. Similarly, a transgenic “knockout mouse” or other animal, is one that comprises cells containing a disrupted gene or allele.

Library: In this description, “library” denotes an integral collection of two or more constituents. A constituent means “an essential part” of the library. A constituent of a library may be a cell or a nucleic acid. For instance, in addition to a cell library, a library may contain a collection of constructs, polynucleotides or RNA molecules. A library may contain a collection of selected drugs or compounds. A library may comprise an integral collection of “pooled” constituents physically present in one vessel. Alternatively, a library may be an integral collection of constituents produced by the inventive methodology that are stored separately from one another.

Marker sequence: A “marker sequence” refers to either a cell selection marker sequence or a reporter marker sequence. A selection marker sequence encodes a selection marker and may be a host cell selection marker or a target cell selection marker. A reporter marker sequence encodes a reporter marker.

Naturally occurring: The term “naturally occurring” connotes to the fact that the object so qualified can be found in nature and has not been modified by human intervention. Thus, a nucleotide sequence is “naturally occurring” if it exists in nature and has not been modified by human intervention. If a polynucleotide is naturally occurring, the nucleotide sequence of the polynucleotide also is “naturally occurring.” Likewise, if a genome of a cell is “naturally occurring,” the nucleotide sequence of the genome is “naturally occurring.”

Nucleic acid: DNA and RNA molecules are examples of nucleic acids. Thus, a vector, a plasmid, a construct, a polynucleotide, an mRNA or a cDNA are all examples of a nucleic acid.

Obtaining a polynucleotide: A polynucleotide may be “obtained” by performing steps to physically separate the polynucleotide from other nucleic acids, such as from a cell genome. Alternatively, a polynucleotide may be “obtained” from a nucleic acid template by performing a PCR reaction to produce specific copies of the polynucleotide. Further still, a polynucleotide may be “obtained” by designing and chemically synthesizing the polynucleotide using nucleotide sequence information, such as that available in databases.

Operably linked: The term “operably linked” refers to a juxtaposition of genetic elements in a relationship permitting them to function in their intended manner. Such elements include, for instance promoters, regulatory sequences, polynucleotides of interest and termination sequences, which when “operably linked” function as intended. Elements that are “operably linked” are also “in frame” with one another.

Origin of replication: refers to a sequence of DNA at which replication is initiated.

Polynucleotide library: A polynucleotide library is an integral collection of at least two polynucleotides.

Precedent cell: If the genome of a cell is the source of the genome, or part thereof, of another cell, then the former cell is a precedent cell of the latter. For instance, a cell is a precedent of its clones.

Predetermined fashion: The phrase “predetermined fashion” is used here to connote the deliberate establishment of criteria by which to arrange or categorize elements of an assemblage. An array arranged “in predetermined fashion,” for instance, means that the collection of elements that constitutes the array (see definition above) reflects a known, non-random arrangement, such that any molecular differences that exist between such elements are translated into a spatial context. For example, a cell may differ from other cells placed into an array, “in predetermined fashion,” if it is selected under certain criteria prior to its placement into the array. For instance, a cell may be selected for placement into an array based upon its cell type, the nature of the gene that is disrupted by a construct, or by the number of gene alleles that have been disrupted. Indeed, the location of a cell in an array is a criteria that also can be established “in predetermined fashion.” In another embodiment, a “predetermined fashion” may entail the location of a cell in an array for the purpose of exposing the cell to a testing environment (as opposed to, for example, locating the cell for the purposes of storage). In a preferred embodiment, the testing is a comparative testing to determine the effect of gene or allele disruption on the phenotype of the cell.

Random insertion: The term “random insertion” refers to the process by which a nucleic acid is integrated into an unspecified region of a genome or DNA preparation.

Regulatable gene: A “regulatable gene” is a gene or polynucleotide sequence whose transcription is modified or whose resultant mRNA transcript is degraded such that the transcript is not transcribed to produce a complete protein as encoded by the gene or polynucleotide sequence. A regulatable gene may be one whose mRNA, while intact, is not translated by the host cell enzymes. In general, a regulatable gene is one that permits its expression at specific times or under specific conditions. For instance, a regulatable gene is one which is driven by an inducible promoter.

Splice donor sequence: A segment of DNA at the 5′ end of an intron that facilitates excision and splicing reactions.

Splice acceptor sequence: A segment of DNA at the 3′ end of an intron that facilitates excision and splicing reactions.

Target cell: A target cell is a cell that whose gene expression is to be or has been altered, preferably by being transformed by a nucleic acid or a construct. In another embodiment, the gene expression is altered by a molecule, such as a chemical agent. Preferred target cells are eukaryotic cells, such as yeast, fungi cells, plant cells, animal cells, mammalian cells, human cells, endothelial cells, epithelial cells, islets, neurons, mesothelial cells, osteocytes, lymphocytes, chondrocytes, hematopoietic cells, immune cells, cells of the major glands; or organs, such as the lung, heart, stomach, pancreas, kidney, skin; exocrine and/or endocrine cells; embryonic and other stem cells, fibroblasts, or tumorigenic cells.

Termination sequence: A polynucleotide sequence, that stops or otherwise prevents the transcription of a region of a genome is known, herein, as a termination sequence. A termination sequence may be, for instance, a polyadenylation sequence, but any sequence that is capable of inhibiting transcription may be used in the context of the instant invention.

Transcribable region: Transcription is the formation of an RNA molecule upon a DNA template by complementary base-pairing. Thus, a transcribable region, represents a DNA template from which an RNA transcript can be generated. Preferably, a transcribable region is a DNA sequence that encodes a protein product. Thus, a transcribable region may be a gene or similar coding region.

Trap construct: A “trap construct” is a construct containing functional elements that facilitate the integration of either its entire sequence or a part of it, into a cell genome, or into any DNA preparation. Such elements include “splice acceptor” and “splice donor” nucleotide sequences. A “trap construct” may be designed to integrate into any part of a gene. In this regard, a “trap construct” may be a “promoter trap,” an “exon trap,” or a “3′-trap” construct. Alternatively, a “trap construct” may integrate into non-transcribable region of a genome. A non-transcribable region is a region that does not encode a gene product, such as a polypeptide.

Upstream: A polynucleotide sequence in a construct is regarded as being upstream or 5′ to a second polynucleotide sequence in the construct, if the 3′ end of the former sequence is located before the 5′ end of the latter sequence.

Vessel: A “vessel” is any structure that is useful in containing a biological substance, such as nucleic acid or cells. For instance, a vessel may be a test tube, an “Eppendorf” tube, a petri dish, a microscope slide or a well.

Well: A “well” is a structure into which a substance, such a liquid, may be contained. A well may be one of an integral collection of wells that constitute an array. The wells of such an array may, or may not, be fixed or attached to one another.

A. Disruption and Identification of Genes in Cells

The present invention provides materials and methods by which the expression of a gene can be modulated, mutated, “knocked-out,” or otherwise disrupted. Furthermore, genomic or cDNA fragments of the disrupted gene or allele can be readily recovered and sequenced to identify the disrupted allele or gene.

In one embodiment, the present invention uses constructs inserted into genomic DNA of cells to disrupt at least one allele of a gene in the provider cell genomic DNA. Cells that have a single allele of a gene disrupted by insertion of a construct have become single copy knockouts. It is an aspect of the instant invention to provide an array of single copy knockout cells. This particular cell can be targeted again by a homologous recombination vector that targets different alleles of the originally disrupted gene allele, so as to produce multiple-allele knockout cells. Alternatively, multiple-allele knockout cells can be produced by introducing a second trap vector to the single copy knockout cells. Preferably, multiple-allele knockout cells can be produced by introducing a homologous recombination vector to a target cell and producing a single copy knockout cell followed by the introduction of a trap vector or second homologous recombination vector. It is an aspect of the instant invention to provide an array of multiple-allele knockout cells.

The integrated construct may be recovered with a portion of flanking genomic DNA and/or cDNAs derived from mRNA transcripts of at least portions of the construct and flanking genomic DNA (“recovered polynucleotide”). Accordingly, a cell in which recovered polynucleotide are isolated is known herein as a provider cell. These recovered polynucleotides can be sequenced and their identity confirmed. The recovered polynucleotides also may be used directly as homologous recombination vectors and replicated in host cells. Cells into which at least a portion of the recovered polynucleotide is inserted are known, herein, as target cells.

Preferred provider or target cells are eukaryotic cells. A more preferred provider or target cell is a mammalian cell, such as a murine or human cell. The target cell may be a somatic cell or a germ cell. The germ cell may be a stem cell, such as embryonic stem cells (ES cells), including murine embryonic stem cells. The provider or target cell may be a non-dividing cell, such as a neuron, or preferably, the provider or target cell can proliferate in vitro under certain culturing conditions.

For instance, the provider or target cell may be chosen from commercially available mammalian cell lines—see the catalogue of ATCC Cell Lines and Hybridomas, American Type Culture Collection, 10801 University Boulevard, Manassas, Va. USA 20110-2209. A provider or target cell also may be any type of diseased cells, including cells with abnormal phenotypes that can be identified using biological or biochemical assays. For instance, the diseased cells may be tumor cells, such as colon cancer cells or Kras transformed colon cancer cells.

A host cell of the present invention preferably is different from the target cell. Suitable host cells may be non-mammalian eukaryotic cells such as yeast or, preferably, are prokaryotic cells, such as bacteria. For instance, the host cell may be a strain of E. coli.

Provider cells in which a trap construct has been inserted can be selected by techniques described herein and/or polynucleotides that flank the inserted trap construct may be recovered from the provider cells. For instance, recovery of polynucleotides may be achieved from reverse transcription of messenger RNAs (mRNA) derived from the disrupted genes, or from genomic DNA fragments that comprise both the trap construct and part of the genomic DNA.

The recovered polynucleotide also can be introduced into host cells. The host cells can be selected for proper transfection by the techniques described herein and/or replicate the recovered polynucleotide. After replication, the nucleotide sequence of the recovered provider cell genomic fragments can then be determined, enabling the flanking genomic DNA fragments to be associated with a larger portion of the provider cell genome, thereby identifying the location and identity of the trap construct insertion by comparison to the known genomic DNA sequence of the provider cell.

If the location of the trap construct insert is determined, this information can be used to create homologous recombination vectors specific to a sequence of the provider cell genome as described herein. Alternatively, if the location of the trap construct insert is not determined, the recovered polynucleotide can be used to create homologous recombination vectors as described herein. However, it is a concept of the instant invention that a genomic or mRNA fragment that contains a gene disrupted by a trap construct, itself, can be used as a homologous recombination vector. That is, upon fragmentation of the genome by restriction nucleases, shearing or by other mechanical forces, the fragment which contains a trap construct, or a portion thereof, can be recircularized and used directly as a homologous recombination vector. Thus, the instant invention envisions the use of a trap construct, or a portion thereof, that is flanked by genomic DNA sequences, as a homologous recombination vector.

Accordingly, there is no need to design genespecific nucleotides or fragments to ligate into a preexisting homologous recombination vector, since those sequences are already present in the trapped genomic fragment. Preferably, the trap construct inserted into the genome does not contain restriction recognition sites that are used to digest and fragment the targeted genome. In this way, the trap construct remains intact and flanked at both the 5′ and 3′ ends with genomic DNA. Nevertheless, the instant invention also envisions the ligation of a trap construct that contains only a 5′ flanking genomic segment with another trap construct that contains a 3′ flanking genomic segment, such that, together, a homologous recombination vector can be formed.

The homologous recombination vectors can be used to create single copy or multiple copy knockouts of target cells. These multiple copy knockout cells are valuable in evaluating the therapeutic or diagnostic utilities of genes inactivated in these cells.

Moreover, the recovered polynucleotide can be used to prepare polynucleotide arrays and polynucleotide libraries, that comprise the flanking genomic or cDNA regions of the recovered polynucleotide. In the polynucleotides libraries, each polynucleotide may represents a disrupted gene. The cells in which the genes are disrupted also compose a library, in which each cell has at least one allele of a gene disrupted by a trap construct or homologous recombination construct introduced to the provider or target cells. The present invention therefore establishes a way to correlate cells in a cell library, disrupted cellular genes, and polynucleotides comprising part of the disrupted genes. This one-to-one correlation enables a convenient way to select therapeutically relevant genes from the plethora of genes discovered by means of genomics technologies.

The recovered polynucleotide can be introduced into a host cell. The recovered polynucleotide can be replicated by the host cell, and/or properly transfected host cells can selected by techniques described below.

In a preferred embodiment, the trap construct and homologous recombination constructs may include combinations of (i) an origin of replication (ii) cell selection marker sequences, (iii) splice acceptor sequence, (iv) splice donor sequence, (v) termination sequence, (vi) internal ribosomal entry sequence (IRES), (vii) promoter sequences, (viii) translation initiation sequences, (ix) recombinase recoginition sites, and other functional elements.

An origin of replication is capable of initiating DNA synthesis in a suitable host cell. Preferably, the origin of replication is selected based on the type of host cell. For instance, it can be eukaryotic (e.g., yeast) or prokaryotic (e.g., bacterial) or a suitable viral origin of replication may be used. Preferably, an origin of replication is capable of initiating DNA synthesis in the host cell but does not function in the provider or target cell.

In a preferred embodiment, a selection marker sequence can be used to eliminate provider cells in which a trap construct has not been properly inserted, to eliminate host cells in which recovered DNA has not been properly transfected, or to eliminate target cells in which trap constructs and/or homologous recombination vectors have not been properly inserted.

A selection marker sequence can be a positive selection marker reporter marker or negative selection marker. Selection marker sequences can also be used in combination with “selection switches” as described herein.

Positive selection markers permit the selection for cells in which the gene product of the marker is expressed. This generally comprises contacting cells with an appropriate agent that, but for the expression of the positive selection marker, kills or otherwise selects against the cells. For suitable positive and negative selection markers, see Table I in U.S. Pat. No. 5,464,764.

Examples of selection markers also include, but are not limited to, proteins conferring resistance to compounds such as antibiotics, proteins conferring the ability to grow on selected substrates, proteins that produce detectable signals such as luminescence, catalytic RNAs and antisense RNAs. A wide variety of such markers are known and available, including, for example, the neomycin resistance (neo) marker (Southern & Berg, J. Mol. Appl. Genet. 1: 327-41 (1982)), the hygromycin resistance (hyg) marker (Te Riele et al., Nature 348:649-651 (1990)), the thymidine kinase (tk), the hypoxanthine phosphoribosyltransferase (hprt), and the bacterial guanine/xanthine phosphoribosyltransferase (gpt), which permits growth on MAX (mycophenolic acid, adenine, and xanthine) medium. See Song et al., Proc. Nat'l Acad. Sci. U.S.A. 84:6820-6824 (1987). Other selection markers include histidinol-dehydrogenase, chloramphenicol-acetyl transferase(CAT), dihydrofolate reductase (DHFR), β-galactosyltransferase and fluorescent proteins such as the Green Fluorescent Protein (GFP) isolated from the bioluminescent jellyfish Aequorea victoria.

Expression of a fluorescent protein can be detected using a fluorescent activated cell sorter (FACS). Expression of β-galactosyltransferase also can be sorted by FACS, coupled with staining of living cells with a suitable substrate for β-galactosidase. A selection marker also may be a cell-substrate adhesion molecule, such as integrins which normally are not expressed by the mouse embryonic stem cells, miniature swine embryonic stem cells, and mouse, porcine and human hematopoietic stem cells. For mammalian cell selection markers, see chapter 16 of Sambrook et al. Target cell selection marker can be of mammalian origin and can be thymidine kinase, aminoglycoside phosphotransferase, asparagine synthetase, adenosine deaminase or metallothionien. The cell selection marker can also be neomycin phosphotransferase, hygromycin phosphotransferase or puromycin phosphotransferase, which confer resistance to G418, hygromycin and puromycin, respectively.

Suitable prokaryotic and/or bacterial selection markers include proteins providing resistance to antibiotics, such as kanamycin, tetracycline, and ampicillin. A suitable fusion protein capable of conferring selectable traits to both a prokaryotic host cell and a mammalian target cell includes a fusion protein of blasticidin S deaminase (bsd), cytidine deaminase (codA) and uracil phosphoribosyltransferase (upp) (bsdS:codA::upp).

Negative selection markers permit the selection against cells in which the gene product of the marker is expressed. In some embodiments, the presence of appropriate agents causes cells that express “negative selection markers” to be killed or otherwise selected against. Alternatively, the expression of negative selection markers alone kills or selects against the cells.

Such negative selection markers include a polypeptide or a polynucleotide that, upon expression in a cell, allows for negative selection of the cell. Illustrative of suitable negative selection markers are (i) herpes simplex virus-thymidine kinase (HSV-TK) marker, for negative selection in the presence of any of the nucleoside analogs acyclovir, gancyclovir, and 5-fluoroiodoamino-Uracil (FIAU), (ii) various toxin proteins such as the diphtheria toxin, the tetanus toxin, the cholera toxin and the pertussis toxin, (iii) hypoxanthine-guanine phosphoribosyl transferase (HPRT), for negative selection in the presence of 6-thioguanine, (iv) activators of apoptosis, or programmed cell death, such as the bc12-binding protein (BAX), (v) the cytidine deaminase (codA) gene of E. coli. and (vi) phosphotidyl choline phospholipase D. For example, see Karreman, Gene 218: 57-61 (1998).

A reporter marker is a molecule, including polypeptide as well as polynucleotide, expression of which in a cell confers a detectable trait to the cell. Preferred reporter markers include, but are not limited to, chloramphenicol-acetyl transferase(CAT), β-galactosyltransferase, horseradish peroxidase, luciferase, alkaline phosphatase, and fluorescent proteins such as the Green Fluorescent Protein (GFP) isolated from the bioluminescent jellyfish Aequorea Victoria.

In accordance with the present invention, the selection marker usually is selected based on the type of the cell undergoing selection. For instance, it can be eukaryotic (e.g., yeast), prokaryotic (e.g., bacterial) or viral. In such an embodiment, the selection marker sequence is operably linked to a promoter that is suited for that type of cell.

In another embodiment, more than one selection marker is used. In such an embodiment, selection markers can be introduced wherein at least one selection marker is suited for one or more of provider, target or host cells.

In such an embodiment, the marker sequence of the promoter trap construct can be a target cell selection marker sequence, and the promoter trap construct further comprises a host cell selection marker sequence.

In a preferred embodiment, the host cell selection marker sequence and the target cell selection marker sequence are within the same open-reading frame and are expressed as a single protein. For example, the host cell and target cell selection marker sequence may encode the same protein, such as blasticidin S deaminase, which confers resistance to Blasticidin for both prokaryotic and eukaryotic cells. The host cell and the target cell marker sequence also may be expressed as a fusion protein. In another embodiment, the host cell and the target cell selection marker sequence are expressed as separate proteins.

Preferably, the splice acceptor site comprises a pyrimidine-rich region, preceding the dinucleotide AG. For instance, a suitable splice acceptor site may be NTN(TC)(TC)(TC)TTT(TC)(TC)(TC)(TC)(TC)(TC)NCAGG.

An example of a suitable splice donor site is NAGGT(AG)AGT.

A typical transcriptional termination sequence includes the polyadenylation site (poly A site). A preferred poly A site is the SV40 poly A site, described in the Invitrogen 1996 Catalogue.

In one embodiment of the present invention, the trap construct or homologous recombination construct also comprises an internal ribosome binding site (IRES), which may improve the translation of a downstream open-reading frame, such as a target cell selection marker sequence or a reporter marker sequence. The IRES site can be located 3′ to the splice acceptor site and 5′ to the marker sequence and may be a mammalian internal ribosome entry site, such as an immunoglobulin heavy chain binding protein internal ribosome binding site. In one embodiment, the IRES sequence is selected from encephalomyocarditis virus, poliovirus, piconaviruses, picorna-related viruses, and hepatitis A and C. Examples of suitable IRES sequences can be found in U.S. Pat. No. 4,937,190, in European patent application 585983, and in PCT applications WO9611211, WO9601324, and WO9424301, respectively.

A promoter can be selected based on the type of provider, host or target cell. Suitable promoters include but are not limited to the ubiquitin promoters, the herpes simplex thymidine kinase promoters, human cytomegalovirus (CMV) promoters/enhancers, SV40 promoters, β-actin promoters, immunoglobulin promoters, regulatable promoters such as metallothionein promoters, adenovirus late promoters, and vaccinia virus 7.5K promoters. The promoter sequence also can be selected to provide tissue-specific transcription.

In another embodiment, a trap construct comprises a translational initiation sequence or enhancer, such as the so-called “Kozak sequence” (Kozak, J. Cell Biol. 108: 229-41 (1989)) or “Shine-Delgarno” sequence. These sequences may be located 3′ to an IRES site but 5′ to a marker sequence.

In another embodiment, termination/stop codon(s) in one or more reading frames are added to the 3′ end of the target or host cell selection marker sequences or the reporter marker sequence, such that translations of these marker sequences, if they encode polypeptides, are terminated at the stop codon(s). Stop codon(s) also may be added at the 5′ side of the marker sequences.

In a preferred embodiment, a trap construct comprises, in the 5′ to 3′ order, a splice acceptor site, an origin of replication, an IRES sequence, a target cell selection marker sequence, and a poly A site. The promoter trap construct also may comprise, in the 5′ to 3′ order, a promoter capable of transcribing a downstream sequence in a host cell but not in a target cell, a Shine-Dalgarno sequence and a host cell selection marker sequence. The Shine-Dalgarno sequence, the host cell selection marker sequence and the promoter are located between the 5′ end of the splice acceptor site and the 3′ end of the poly A site. For instance, it can be located between the 3′ end of the splice acceptor site and the 5′ end of the IRES site. In another embodiment, the poly A site is replaced with a splice donor site. In yet another embodiment, the target cell selection marker and the host cell selection marker are expressed as a single protein.

Recombinase recognition sites may be used for insertion, inversion or replacement of DNA sequences, or for creating chromosomal rearrangements such as inversions, deletions and translocations. For example, two recombinase recognition sites in a trap construct or homologous recombination construct may be in the same orientation, to allow removal or replacement of the sequence between these two recombinase recognition sites upon contact with a recombinase. Two recombinase recognition sites may also be incorporated in opposite orientations, to allow the sequence between these two sites to be inverted upon contact with a recombinase. Such an inversion can be used to regulate the function of a trap construct or homologous recombination construct. Therefore, changing the orientation of the construct may switch on or off the construct's effect.

In one embodiment, a trap construct or homologous recombination construct with recombinase recoginition sites is first incorporated into the genome of a target cell, for example via random insertion. A recombinase recognizing the recombinase recoginition sites then is introduced into the provider or target cell to regulate the function of the trap construct or homologous recombination construct. In another embodiment, recombinase recognition sites are first incorporated into the genome of a provider or target cell, and then, a trap construct or homologous recombination construct with the same recombinase recoginition sites may be introduced into the provider or target cell, together with a recombinase capable of recoginizing the recombinase recoginition sites. The recombinase may mediate insertion of the trap construct or homologous recombination construct into the genome of a target cell via the already incorporated recombinase recoginition sites.

Examples of suitable recombinase recognition sites include frt sites and lox sites, which can be recognized by flp and cre recombinases, respectively. See U.S. Pat. Nos. 6,080,576, 5,434,066 and 4,959,317. Other elements, such as transposable elements and recombinase recognition sequences, also may be added to the trap construct used in the present invention to improve the insertion or other functions of the construct.

All of the above-described functional elements can be used in any combination to produce a suitable trap construct or homologous recombination vector. Below are non-limiting examples of the trap constructs and other techniques. In its simplest form, the trap construct can be a genomic DNA construct comprising an origin of replication or host selection marker and/or target selection marker. The construct may also include a promoter. Examples of trap constructs include, but are not limited to, genomic DNA trap constructs, promoter trap constructs, 3′ trap constructs, or exon trap constructs. Stanford et al., Nature Reviews: Genetics, vol. 2, 756-768, 2001, describes different types of trap constructs that can be used in the context of the instant invention.

In one embodiment, an origin of replication and/or host cell selection marker can be upstream or downstream of the splice acceptor sequence.

In a preferred embodiment, the recovered polynucleotide can comprise genomic DNA or mRNA transcripts of genomic DNA flanking both ends of at least a part of the inserted trap construct (or be manipulated to this result). This recovered polynucleotide can be used to produce homologous recombination vectors.

In a preferred embodiment, the origin of replication and/or host cell selection marker is downstream of the splice acceptor sequence and/or between the splice acceptor and any termination sequence or splice donor sequence. In such embodiments, a trap construct flanked by transcribed genomic DNA can be isolated and a plasmid produced. Such plasmids can be transfected to host cells and replicated.

The present invention also envisions the incorporation of a trap construct into an in vitro preparation of genomic DNA. That is, the invention is not limited to the insertion of a construct only into an intact genome of a cell, but also encompasses insertion into an isolated preparation of genomic DNA preparations. Thus, DNA from a cell can be prepared according to standard techniques and used as a template into which a trap construct can be inserted. The genomic preparation then may be fragmented, the fragments circularized and used to transfect a host cell. Accordingly, only those fragments containing the trap construct with a suitable selectable marker can be identified.

The instant invention also is not limited to the location in which a trap construct of the instant invention is inserted into a cell genome. That is, a construct may be inserted into a non-transcribed region of a target genome and not necessarily into a gene of that target cell genome. Accordingly, non-transcribed regions of a genome may be disrupted according to the instant invention.

The following trap constructs are examples of those that may be used in the instant invention:

i. Promoter Trap Construct

In one embodiment, a promoter trap construct comprises (i) a splice acceptor sequence, (ii) a selection marker sequence appropriate for the cell in which the promoter trap construct is inserted and (iii) an origin of replication and/or a host cell selection marker.

Preferably, the promoter trap construct comprises (i) a splice acceptor sequence, (ii) a selection marker sequence appropriate for the cell in which the promoter trap construct is inserted and (iii) an origin of replication and (iv) a host cell selection marker. In such an embodiment, elements (ii) and (iv) can have the same open reading frame or be the same protein.

In another embodiment, the present invention further comprises any or a combination of an IRES sequence, a transcriptional termination sequence and/or a splice donor sequence. Preferably, the IRES sequence is upstream of one or more of the marker sequence(s). Likewise, the termination sequence and/or a splice donor sequence is preferably downstream of one or more of the marker sequence(s).

For example, in one embodiment, the promoter trap construct comprises a transcriptional termination sequence along with the splice acceptor site, a marker sequence and origin of replication. In another embodiment, the promoter trap construct comprises a splice donor site along with the splice acceptor site, a marker sequence and origin of replication.

Preferably, the origin of replication and the marker sequence are located upstream to the 3′-end of the termination sequence or the splice donor site. In one embodiment, the origin of replication and a marker sequence are located downstream to the 3′-end of the splice acceptor site and upstream to the 5′-end of the termination sequence or the splice donor site.

In yet another embodiment, the origin of replication in the promoter trap construct may be located either downstream to a marker sequence, or between the splice acceptor site and a marker sequence. It also can be located within a marker sequence, provided that it does not significantly interfere with the intended function of the marker encoded by the marker sequence. In another embodiment, the origin of replication is located downstream to a marker sequence and upstream to the transcriptional termination sequence/splice donor site.

In one embodiment, the marker sequence of the promoter trap construct is either a target cell selection marker sequence or a reporter marker sequence. In accordance with the present invention, a selection marker, such as a target cell selection marker and a host cell selection marker, is a molecule that confers a selectable trait to a target or a host cell, respectively. A selection marker may be, for example, a polypeptide or a polynucleotide. Methods of selection include but are not limited to antibiotic, colorimetric, enzymatic, and fluorescent selection. See, for example, U.S. Pat. Nos. 5,464,764 and 5,625,048.

ii. 3′ Gene Trap Construct

In accordance with another aspect of the present invention, the trap construct is a 3′ gene trap construct which comprises a transcriptional initiation sequence, an origin of replication and a marker sequence. The origin of replication and the marker sequence are located downstream to the 5′-end of the transcriptional initiation sequence. The marker sequence can be either a target cell selection marker sequence or a reporter marker sequence.

In a preferred embodiment, the 3′ gene trap construct comprises a splice donor site. The origin of replication and the marker sequence are upstream to the 3′-end of the splice donor site. Preferably, the origin of replication is exogenous to either the transcriptional initiation sequence or the splice donor site, and can be located either downstream or upstream to the marker sequence. The origin of replication and the marker sequence may be located downstream to the 3′-end of the transcriptional initiation sequence and upstream to the 5′-end of the splice donor site. Both the origin of replication and the marker sequence have the same general features as the origin of replication and the marker sequence of the promoter trap construct, respectively.

In another embodiment, the 3′ gene trap construct comprises between about 1 and about several thousand bases of intron sequence that are adjacent and 3′ to the splice donor site. This additional intron sequence may improve the splicing efficiency of the splice donor site. Moreover, the expressible open reading frame sequence 5′ to the splice donor site, for example, the target cell selection marker sequence, may be selected so as to improve the splicing efficiency of the splice donor site. See U.S. Pat. No. 6,080,576.

In yet another embodiment, the marker sequence is a target cell selection marker sequence, and the 3′ gene trap construct further comprises a host cell selection marker sequence located downstream to the 5′-end of the transcriptional initiation sequence and upstream to the 3′-end of the splice donor site. Preferably, the host cell selection marker sequence is located downstream to the 3′-end of the transcriptional initiation sequence and upstream to the 5′-end of the splice donor site. The host cell and target cell selection marker sequence have the same general features as the host cell and target cell selection marker sequence of the promoter trap construct, respectively. For example, the host cell selection marker and target cell selection marker can be expressed either as separate proteins or as a single protein.

An IRES site, a translational initiation sequence or enhancer such as the Kozak sequence, and/or a Shine-Dalgarno sequence may be incorporated at the 5′ side of the target cell or host cell selection marker sequence, in a manner similar to the construction of the promoter trap construct. Likewise, termination/stop codon(s) can be added to the 3′ or 5′ side of the target cell or host cell selection marker sequence. These additional elements or sequences preferably are located between the 5′ end of the transcriptional initiation sequence and the 3′ end of the splice donor site.

In a preferred embodiment, the 3′ gene trap construct comprises a negative selection marker sequence located 3′ to the splice donor site. When the 3′ gene trap construct of the above preferred embodiment is inserted into a non-transcribable region of the genome of a target cell, but is still capable of being transcribed and processed into a mRNA, the negative selection marker also is expressed therewith, killing the target cell. But when the 3′ gene trap construct is inserted into a transcribable genomic sequence, such as an exon or intron of an expressible gene, the negative selection marker sequence may be spliced out, by virtue of the splice donor site located 5′ to the negative selection marker sequence. The removal of the negative selection marker would possibly allow the target cell to survive selection directed against the negative selection marker. Consequently, the presence of the negative selection marker sequence can reduce the incidence of a false-positive selection of a target cell in which a 3′ gene trap construct is inserted into a non-transcribable genomic sequence and yet is transcribed and processed into a mRNA transcript.

In another preferred embodiment, the 3′ gene trap construct comprises, in the 5′ to 3′ order, a transcriptional initiation sequence capable of transcribing the downstream sequence in a target cell, an origin of replication, an IRES site, a target cell selection marker sequence, and a splice donor site. The 3′ gene trap construct also may comprise, in the 5′ to 3′ order, a promoter capable of transcribing a downstream sequence in a host cell but in a target cell, a Shine-Dalgarno sequence and a host cell selection marker sequence. These sequences may be located downstream to the transcription initiation sequence but upstream to the splice donor site. In one embodiment, the host cell and target cell marker sequence are expressed as a single protein. In another embodiment, the 3′ gene trap construct further comprises a negative selection marker sequence located 3′ to the splice donor site.

iii. Exon Trap Construct

According to one aspect of the present invention, an exon trap construct comprises an origin of replication and a marker sequence which have the same general features as the corresponding sequences in the promoter trap construct. Promoter trap constructs and 3′ gene trap constructs described above are examples of exon trap constructs. The origin of replication may be either upstream or downstream to the marker sequence. In one embodiment, the exon trap construct does not comprise either a splice acceptor site or a transcriptional initiation sequence.

In a preferred embodiment, the marker sequence is a target cell selection marker sequence, and the exon trap construct further comprises a host cell selection marker sequence. The target cell and host cell selection marker sequences have the same general features as those in the promoter trap construct. An IRES, a translational initiation sequence or enhancer such as the Kozak sequence, a Shine-Dalgarno sequence and/or a series of termination/stop codons can be added in the exon trap construct, in a manner similar to the construction of the promoter trap construct.

In another embodiment, the exon trap construct comprises a transcriptional termination sequence, such as a poly A site, or a splice donor site. The transcriptional termination sequence or the splice donor site is located downstream to the origin of replication, the target cell selection marker sequence and the host cell selection marker sequence.

B. Trap Construct Comprising Recombinase Recognition Sites

In one embodiment, a trap construct comprises two recombinase recognition sites, which are preferably located at the 5′ and 3′ ends of the construct. The trap construct may be a promoter trap construct, a 3′ gene trap construct, or an exon trap construct. In another embodiment, the two recombinase recognition sites are located at the 5′ and 3′ ends of an element of the trap construct. For instance, two lox sites or two frt sites may be located at the 5′ and 3′ ends of the marker sequence, which can be either a target cell selection marker sequence or a reporter marker sequence.

C. Polynucleotide Libraries and Polynucleotide Arrays

Based upon the information provided herein, numerous polynucleotide and cell libraries can be produced. These libraries include, but are not limited to, libraries of (i) trap constructs, (ii) single copy knockouts (iii) single copy knockouts produced by insertion of trap construct(s) into the cell's genomic DNA, (iv) recovered polynucleotides and/or cDNAs thereof, (v) genomic DNA isolated from recovered polynucleotides, (vi) probes and primers to (v), (vii) probes and primers to genomic DNA in proximity to v above (viii) circularized recovered polynucleotides, (ix) homologous recombination vectors, (x) single copy knockouts produced by insertion of homologous recombination vectors into a cell's genomic DNA and (xi) multiple copy knockouts.

In (vii) above, “in proximity to” means a polymerase chain reaction (PCR) primer may be designed upstream or downstream of the recovered polynucleotide sequence such that it may be used, in conjunction with a primer designed within the recovered polynucleotide, to generate a product that can be repeatedly amplified. This technique can be used to verify homologous recombination.

A trap construct of the present invention can be used to trap genes in the genome of any type of target cells. A trap construct can be introduced into a target cell by any methods as appreciated in the art, including but not limited to, electroporation, viral infection, retrotransposition, microinjection, lipofection, liposome-mediated transfection, calcium phosphate precipitation, DEAE-dextran, and ballistic or “gene gun” penetration. For the use of a viral vector to introduce a vector into a target cell, see U.S. Pat. Nos. 6,080,576 and 5,922,601.

In accordance with one aspect of the present invention, a promoter trap construct is introduced into the genome of a target cell, for example, via random insertion. Special chemicals may be used to increase the activity in certain regions of the genome so as to promote integration of the trap construct. The promoter trap construct may be inserted into a transcriptionally active genomic sequence which encodes, for example, an actively transcribed gene. The construct sequence that is 3′ to the splice acceptor site of the construct, together with part of the transcriptionally active genomic sequence, may be transcribed and then processed into a mRNA, from which the target cell selection/reporter marker encoded by the marker sequence of the construct may be expressed. In a preferred embodiment, the marker sequence encodes a target cell selection marker, such that the target cell comprising the trap construct can be selected for the selectable trait conferred by the selection marker. In yet another preferred embodiment, the promoter trap construct further comprises a host cell selection marker sequence.

In a preferred embodiment, the promoter trap construct comprises a splice acceptor site 5′ to other elements in the construct. These other elements may include a host cell selection marker sequence, an origin of replication and a target cell selection marker sequence. Preferably, the promoter trap construct also comprises a transcriptional termination sequence, or a splice donor site, that is downstream to other elements of the construct. The host cell and target cell selection marker may be expressed as a single protein. When the promoter trap construct is inserted into an actively transcribed gene of a target cell, the exon(s) of the gene that are 5′ to the splice acceptor site of the construct, together with the origin of replication, the host selection marker sequence and the target cell selection marker sequence of the construct, may be transcribed and processed into a mRNA. The genomic sequence 3′ to the inserted construct also may be transcribed and processed into the mRNA, for example, if the construct contains a splice donor site but not a transcriptional termination sequence.

Pursuant to another aspect of the invention, a 3′ gene trap construct is incorporated into the genome of a target cell. Selection is effected to identify instances where the construct is inserted within a transcribable genomic sequence, such as a sequence that can be transcribed under certain conditions and has a transcriptional termination sequence. A gene is an example of a transcribable genomic sequence. The construct sequence that is 3′ to the transcriptional initiation sequence of the construct, together with part of the transcribable genomic sequence, may be transcribed and processed into mRNA, from which the target cell selection/reporter marker and/or origin of replication encoded by the marker sequence of the construct can be expressed. In a preferred embodiment, the marker sequence encodes a target cell selection marker and/or origin of replication, and thus the target cell can be selected by the selectable trait conferred by the marker. In another preferred embodiment, the 3′ gene trap construct further comprises a host cell selection marker sequence.

In a preferred embodiment, the 3′ gene trap construct comprises, in the 5′ to 3′ order, a transcriptional initiation sequence, an origin of replication, a host cell selection marker sequence and a target cell selection marker sequence. When the construct is inserted into a transcribable gene of the target cell, the genomic sequence of the gene that are 3′ to the inserted construct, together with the host cell selection marker sequence, the target cell selection marker sequence and the origin of replication of the construct, may be transcribed under control of the transcription initiation sequence of the construct, and processed into mRNA. Preferably, the construct also comprises a splice donor site downstream to the above mentioned elements.

In yet another aspect of the present invention, an exon trap construct is introduced into a target cell and incorporated into its genome. The construct may be inserted into an exon of an actively transcribed gene, so that the construct as well as part of the gene can be transcribed and processed into mRNA, from which the target cell selection/reporter marker encoded by the construct may be expressed. In a preferred embodiment, the marker sequence encodes a target cell selection marker, and thus the target cell can be selected by the selectable trait conferred by the marker. In another preferred embodiment, the exon trap construct further comprises a host cell selection marker sequence.

In one embodiment, the exon trap construct comprises a splice donor site 3′ to the target cell selection marker sequence. Insertion of the construct into an intron of an actively transcribed gene may produce a mRNA, from which the target cell selection marker can be expressed, enabling selection of the target cell.

A polynucleotide that comprises part of the trap construct and part of the disrupted gene may be recovered from the mutated target cell. The identity of the disrupted gene may be subsequently determined, for example, by amplifying and sequencing the recovered polynucleotide.

In one embodiment, a trap construct comprising a target cell selection marker sequence and an origin of replication disrupts an allele of a gene in a target cell. The target cell is selected and multiplied under selection conditions for the target cell selection marker. The mRNAs isolated from the multiplied target cells are subject to 5′ or 3′ RACE protocols, to identify the genomic sequences adjacent to the inserted trap construct.

In a preferred embodiment, the mRNA derived from the disrupted gene is reverse transcribed, so the cDNA thus produced may comprise the origin of replication of the trap construct, as well as part of the disrupted gene. The cDNA then may be circularized and introduced into a suitable host cell in which the origin of replication is capable of starting DNA synthesis. If the trap construct, and therefore the cDNA, further comprises a host cell selection marker sequence and/or origin of replication, the cDNA may be amplified in the host cell under selection conditions for the host cell selection marker. The sequence of the amplified cDNA, including part of the disrupted gene, can be determined using methods as appreciated in the art.

In another embodiment, the trap construct does not comprise a host cell selection marker sequence but may have an origin of replication . A host cell selection marker sequence may be added to the reverse transcribed cDNA, such that the modified polynucleotide comprises both the origin of replication of the trap construct and a host cell selection marker sequence. Preferably, the polynucleotide thus modified is circularized, and then amplified and selected in suitable host cells.

Likewise, in another embodiment, the trap construct comprises a host cell selection marker sequence but not an origin of replication. An origin of replication may be added to the reverse transcribed cDNA, such that the modified polynucleotide comprises both the origin of replication and the host cell selection marker sequence. Preferably, the modified polynucleotide is circularized, and then amplified and selected in host cells.

In yet another embodiment, the reverse transcribed cDNA may be circularized via a linking polynucleotide as used for circularizing the genomic DNA fragments created for making homologous recombination vectors, as described below. The linking polynucleotide may provide an origin of replication or a host cell selection marker sequence that is absent from the trap construct, thus enabling the circularized product to be amplified and selected in suitable host cells.

In one embodiment, the amplified cDNA comprising part of the disrupted gene may serve as an index for the disrupted gene, as well as for the target cell in which the gene is disrupted. Thus, a target cell library and a corresponding polynucleotide library can be created. Each cell in the cell library has at least one allele of a gene disrupted by a trap construct, and each disrupted gene has a corresponding polynucleotide in the polynucleotide library, such that the corresponding polynucleotide comprises part of the sequence of the disrupted gene. This one-to-one correspondence between a cell library and a polynucleotide library is important for functional genomics analysis, where the cell library may be used to evaluate the therapeutic utilities of the disrupted genes. For instance, once the therapeutic effect of a gene is demonstrated using the cell library, the identity of a disrupted gene can be easily determined by reference to, and use of the polynucleotide library.

A polynucleotide library that has a one-to-one correspondence with a cell library may be prepared by other ways. For example, it may be prepared using the polymerase chain reaction (PCR), RACE, or other gene discovery technologies, as one of skill in the art would appreciate, to isolate part of the sequence of the disrupted gene in each cell of the cell library.

In another embodiment of the present invention, the polynucleotide library, in which each polynucleotide comprises part of a disrupted gene, may be used to make polynucleotide arrays representative of the disrupted genes. Each polynucleotide, or fragment thereof, in the library may be spotted onto a suitable medium. Any method for spotting polynucleotides on an array medium may be used. In a preferred embodiment, only a fragment of the disrupted gene is amplified, for example via PCR, from each polynucleotide in the polynucleotide library. The amplified fragment may be isolated and purified, a small amount of which is deposited on an array medium, such as a glass surface, in an array format with each fragment occupying a distinguished position. The deposited fragment is then bonded to the surface of the array medium using standard skill in the art. The polynucleotide arrays according to the present invention may be used, in conjunction with the presently described cell libraries and/or polynucleotide libraries, in functional genomics and target validation studies. For instance, a diseased cell with a diseased phenotype may have a plurality of over-expressed genes. These genes can be identified using a polynucleotide array, when compared to these genes' expressions in normal cells. The corresponding cell in which one of these identified, over-expressed genes is disrupted may be directly selected from the cell library, to evaluate the effect of the disruption of one allele of the gene, or the disruption of any or all alleles of the gene (described below), on the diseased phenotype.

D. Homologous Recombination Vector

A polynucleotide that comprises part of the trap construct and part of the disrupted gene may be isolated from a target cell in which one allele of the gene is disrupted by a trap construct. The isolated polynucleotide may be used to construct a homologous recombination vector to disrupt an allele or alleles of a gene in the target cell. That is, in its most core embodiment, a homologous recombination vector of the instant invention is a trap construct flanked at either one end or both ends by endogenous nucleic acid sequence(s). The latter sequence(s) are capable of initiating a recombination event with similar, if not identical, sequences in a genome of a cell or preparation of DNA.

There are many methods of converting an isolated polynucleotide into a homologous recombination vector. When the isolated polynucleotide has genomic DNA fragments on both ends, the isolated polynucleotide may be suitable as a homologous recombination vector without further manipulation. Likewise, the isolated polynucleotide may be manipulated using standard tools in the art. Such adjustments may include but are not limited to replacing sequences within the isolated polynucleotide, removing sequences from the isolated polynucleotide, and inserting sequences into the isolated polynucleotide. Changes along these lines may include functional elements, such as cell selection markers.

When the isolated polynucleotide has genomic DNA fragment on one end, the isolated polynucleotide may be suitable as a homologous recombination vector without further manipulation. Likewise, the isolated polynucleotide may be manipulated using standard tools in the art. Such adjustments include but are not limited to replacing sequences within the isolated polynucleotide, removing sequences from the isolated polynucleotide, inserting sequences into the isolated polynucleotide. Such changes may include functional elements, such as cell selection markers, etc. Moreover, the isolated polynucleotide may be manipulated by circularizing the isolated polynucleotide for amplification and/or cutting the circularized polynucleotide within the genomic DNA portion to produce an isolated polynucleotide having genomic DNA at both ends or cutting the polynucleotide such that genomic DNA is present at one end.

Accordingly, the present invention provides a method to disrupt any, or all, alleles of a gene in a target cell. The target cells thus made, known as homozygous knockout cells, are useful to evaluate the therapeutic or diagnostic utilities of the inactivated genes, and to screen for compounds that affect the expression and function of the genes.

In one embodiment, each cell in a cell library has one allele of a gene disrupted. Each disrupted gene is represented by a polynucleotide (that comprises part of the disrupted gene) in a polynucleotide library. Each polynucleotide in the polynucleotide library can be used to make a homologous recombination vector directed to the gene represented by the polynucleotide. The homologous recombination vectors thus prepared constitute a homologous recombination vector library. Each vector in the homologous recombination vector library may be used to produce a homozygous knockout target cell, from which a homozygous knockout target cell library is created. Therefore, in certain embodiments the present invention provides a method to make a system comprising a polynucleotide library, a target cell library, a homologous recombination vector library and a homozygous knockout target cell library. Each member in any given library in the system has a corresponding member in any other libraries in the system. This system, together with polynucleotide arrays prepared from the polynucleotide library, is useful to correlate a gene's sequence to it's therapeutic utility, through the use, for example, of the cell libraries in the system.

In one embodiment of the present invention, the homologous recombination vector comprises a trap construct flanked by a first and a second genomic sequence of a target cell. The first and second genomic sequence preferably are part of the same gene. The first and second genomic sequence may be at least about 25 bp or 25-50 bp, 50-100 bp, preferably about 100-200 bp, and more preferably about 300-1000 bp, 1000-2000 bp, 2000-5000 bp, 5000-7000 bp or more than 5000 bp. The first and second genomic sequence may be non-continuous, but preferably continuous, in the genome of the target cell before the gene comprising these sequences is disrupted by the trap construct. The first and second genomic sequence preferably are not continuous in the homologous recombination vector.

As used herein, two nucleotide sequences are continuous if the 3′ end of one nucleotide sequence is covalently linked to the 5′ end of the other nucleotide sequence without any intervening nucleotide residue.

The trap construct in the homologous recombination vector may be any type of trap construct known in the art. Preferably, the trap construct in the homologous recombination vector comprises an origin of replication capable of starting DNA synthesis in a suitable host cell and/or a cell selection marker. The trap construct can be a promoter trap construct, a 3′ gene trap construct, or an exon trap construct. In a preferred embodiment, the trap construct further comprises a host cell selection marker.

The homologous recombination vector can be prepared in various ways. For example, the first and second genomic sequence may be obtained from available genome database or gene expression database for human or other species. The two sequences may be amplified, and then ligated with a trap construct, using methods as appreciated in the art.

In a preferred embodiment, the homologous recombination vector is derived from a target cell in which at least one allele of a gene is disrupted by a trap construct. The trap construct comprises a target cell selection marker sequence and an origin of replication that is capable of starting DNA synthesis in suitable host cells and/or a host cell selection marker. The target cell selection marker sequence is expressed, by virtue of the insertion of the trap construct into the gene, conferring a selectable trait to the target cell. The target cell is then multiplied under selection conditions for the target cell selection marker. Genomic DNAs or DNA fragments are subsequently isolated from the multiplied target cells using methods as appreciated in the art.

The isolated genomic DNAs or DNA fragments may be subject to restriction endonuclease digestion. One or more endonucleases may be used for the digestion. The digestion creates a plurality of genomic DNA fragments, from which the fragment that comprises the trap construct flanked by a first and a second genomic sequence can be identified as described below. The first and second genomic sequence are parts of the gene disrupted by the trap construct.

The genomic DNA fragments produced by restriction endonuclease digestion may be mixed with polynucleotides having compatible 5′ and 3′ ends, such that each genomic DNA fragment can be ligated with one of the polynucleotides. As used herein, these polynucleotides are termed “linking polynucleotides.” The linking polynucleotides may comprise multiple cloning sites at their 5′ and 3′ ends. The ligation products between the genomic DNA fragments and the linking polynucleotides preferably are circular polynucleotides. Either the trap construct or the linking molecule may comprise a host cell selection marker sequence. The ligation products can be introduced into suitable host cells and are selected for the host cell selection marker. Only the ligation product derived from the genomic DNA fragment that comprises the inserted trap construct may be amplified in the host cells, by virtue of the origin of replication comprised in the trap construct.

In one embodiment, the trap construct comprises a host cell selection marker sequence but not an origin of replication, while the linking polynucleotides comprise an origin of replication but not a host cell selection marker sequence. Thus, only the ligation product between a linking polynucleotide and the genomic DNA fragment that comprises the trap construct can be selected and amplified in the host cells, by virtue of the host cell selection marker sequence comprised in the trap construct.

In another embodiment, the trap construct comprises both a host cell selection marker sequence and an origin of replication. The genomic DNA fragments, for example, produced by restriction endonuclease digestion, may be circularized with or without linking polynucleotides. Only the genomic DNA fragment that comprises the trap construct, however, may be selected and amplified in the host cells, by virtue of the host cell selection marker sequence and the origin of replication comprised in the construct.

The selected and amplified ligation product or genomic DNA fragment comprises the trap construct flanked by parts of the genomic sequence of the disrupted gene. The sequence of the disrupted gene may be determined using methods as appreciated in the art. In addition, the selected ligation product or genomic DNA fragment may be used to make a homologous recombination vector, to inactivate the other allele or alleles of the disrupted gene in the target cell.

In one embodiment, the selected ligation product or genomic DNA fragment may be linearized, for example, by restriction endonuclease digestion. The trap construct comprised in the product or fragment may not contain any recognition site for the digestion, so that the digestion does not cut through the trap construct. The product thus linearized comprises the trap construct flanked by two genomic sequences which are parts of the disrupted gene. This product may be used as a homologous recombination vector to make homozygous knockout target cells in which all alleles of the disrupted gene are disrupted. In another embodiment, the linearized product may be incorporated into a vector, such as a viral or retroviral vector, to facilitate homologous recombination in target cells.

In yet another embodiment, a second target cell selection marker sequence, separate from the original target cell selection marker sequence of the trap construct, may be introduced to the homologous recombination vector. For example, in the above described embodiment, the linking polynucleotide may comprise a second target cell selection marker sequence. In a preferred embodiment, the linking polynucleotide also comprises a transcriptional initiation sequence 5′ to the second target cell selection marker sequence, such that the second target cell selection marker sequence can be expressed in the target cell.

The second target cell selection marker sequence in the linking polynucleotide may encode the same target cell selection marker encoded by the original target cell selection marker sequence. Preferably, the second target cell selection marker sequence encodes a different selection marker that confers a selectable trait distinct from that conferred by the original target cell selection marker sequence. More preferably, the second target cell selection marker is a negative selection marker and the original target cell selection marker is a positive selection marker. For example, the second target cell selection marker is HSV-TK and the original target cell selection marker is neomycin phosphotransferase.

In a preferred embodiment, the linking polynucleotide comprises a second target cell selection marker sequence encoding a negative selection marker. The ligation product between the linking polynucleotide and the genomic DNA fragment comprising the trap construct may be amplified in suitable host cells, and linearized, for example, by restriction endonuclease digestion. Preferably, the digestion does not cut through either the trap construct or the second target cell selection marker sequence, such that the linearized product comprises: (1) a cassette, comprising the trap construct flanked by two genomic sequences which are parts of the disrupted gene; and (2) a second target cell selection marker sequence which is located either 5′ or 3′ to the cassette. This linearized product is a preferred homologous recombination vector of the present invention.

In another embodiment, the original target cell selection marker sequence of the trap construct in a homologous recombination vector may be replaced by a new target cell selection marker sequence and/or a reporter marker sequence. Preferably, the new target cell selection marker sequence encodes a different target cell selection marker that confers a different selectable trait than that conferred by the original target cell selection marker. For instance, the trap construct in a homologous recombination vector may have a multiple cloning site (MCS) located at each end of the original target cell selection marker sequence. The 5′-end multiple cloning site may be the same or different from the 3′-end multiple cloning site. The original target cell selection marker sequence may be released from the homologous recombination vector by enzymatic digestion, for example, using restriction endonuclease(s) unique to the multiple cloning sites. To this end, the homologous recombination vector may be first circularized, so that the above-described digestion produces only two fragments. The digestion also may be performed before the homologous recombination vector is linearized during its preparation. The homologous recombinant vector with the original target cell selection marker sequence thus deleted may be ligated to a cassette sequence comprising a new target cell selection marker sequence and/or a reporter marker sequence. The final product, which preferably is circular, may be linearized, and used as a homologous recombination vector.

In yet another embodiment, the original target cell selection marker sequence of the trap construct in a homologous recombination vector may be flanked at both 5′ and 3′ end by a recombinase recognition site, such as the lox site. A cassette that is flanked by the same recombinase recognition site and comprises a new target cell selection marker sequence and/or a reporter marker sequence may be used to replace the original target cell selection marker sequence in the homologous recombination vector, in the presence of a suitable recombinase, such as cre recombinase.

In another preferred embodiment, a homologous recombination vector of the present invention comprises a trap construct flanked by a first and a second sequence. The first and second sequence are homologous to a first and a second genomic sequence of a target cell, respectively. Preferably, the genome of the target cell comprises a gene that is disrupted by a trap construct, and the first and second genomic sequence are parts of the disrupted gene. The first and second genomic sequence may be at least about 50 bp, preferably at least about 100-200 bp, and more preferably at least about 300-1000 bp but generally less than about 15,000 bp. In one embodiment, the first and second sequence are not continuous in the homologous recombination vector, and the first and second genomic sequence are continuous in the genome before the gene is disrupted by the trap construct. In another embodiment, the first and second genomic sequence are not continuous in the genome before the gene is disrupted. The homologous recombination vector of this embodiment may be prepared, for example, by mutating or modifying the first and second genomic sequence in a homologous recombination vector prepared from the genomic DNA fragments of a target cell, using one of the methods described above.

In the present invention, a polynucleotide sequence is homologous to another if the two sequences have at least more than 60%, preferably more than 70%, highly preferably more than 80%, and most preferably more than 90%, sequence identity. In a particular embodiment, a polynucleotide sequence is homologous to another if the two sequences have at least more than 95%-98% sequence identity. Two identical sequences are homologous to each other. “Sequence identify” has an art-recognized meaning and can be calculated using published techniques. See Computational Molecular Biology, Lesk, ed., Oxford University Press, New York, 1988; Biocomputing: Informatics And Genome Projects, Smith, ed., Academic Press, New York, 1993; Computer Analysis Of Sequence Data, Part I, Griffin & Griffin, eds., Humana Press, New Jersey, 1994; Sequence Analysis In Molecular Biology, Von Heinje ed., Academic Press, 1987; Sequence Analysis Primer, Gribskov & Devereux, eds., M. Stockton Press, New York, 1991; Carillo & Lipton, SIAM J. Applied Math. 48:1073 (1988). Methods commonly employed to determine identity or similarity between two sequences include, but are not limited to, those disclosed in Guide To Huge Computers, Bishop, ed., Academic Press, San Diego, 1994, and Carillo & Lipton (1988). Methods to determine identity and similarity are codified in computer programs. Preferred computer program methods to determine identity and similarity between two sequences include, but are not limited to, GCG program package (Devereux et al., Nucleic Acids Research 12(1):387 (1984)), BLASTP, BLASTN, FASTA (Atschul et al., J. Mol. Biol. 215:403 (1990)), and FASTDB (Brutlag et al., Comp. App. Biosci. 6:237-245 (1990)).

Two homologous sequences may hybridize to each other under highly stringent conditions. In the present invention, highly stringent conditions means to hybridize to a filter-bound sequence in a solution containing 6×SSC, 5×Denhardt's reagent, 0.5% SDS, and 100 μg/ml denatured fragment DNA of salmon sperm or calf thymus for 12 hours at 650 C., and then wash in a solution containing 2×SSC and 0.1% SDS for 30 minutes at 250 C., and then wash in a solution containing 0.1×SSC and 0.1% SDS for 10 minutes at 250 C.

Two homologous sequences may hybridize to each other under less stringent conditions. In the present invention, less stringent conditions means to hybridize to a filter-bound latter sequence in a solution containing 3×SSC, 5×Denhardt's reagent, 0.1% SDS, 50 μg/ml denatured fragment DNA of salmon sperm or calf thymus for 12 hours at 500 C., and then wash twice in a solution containing 0.1×SSC and 0.1% SDS for 10 minutes at 250 C.

Any trap construct, including the trap constructs used in the present invention, can be employed to make a homologous recombination vector using the methods described above.

E. Homologous Recombination Vector Comprising a Reporter Marker Sequence

In one embodiment, the original target cell selection marker sequence of the trap construct in a homologous recombination vector of the present invention may be replaced by a reporter marker sequence, using the methods as described above. In a preferred embodiment, the target cell selection marker sequence of the trap construct in a homologous recombination vector is replaced with a polynucleotide comprising a reporter marker sequence and a new target cell selection marker sequence.

In a preferred embodiment, the trap construct in the homologous recombination vector is a promoter trap construct or an exon trap construct, such that the expression of the replaced reporter marker sequence is not controlled by any transcriptional initiation sequence in the homologous recombination vector. Thus, when the homologous recombination vector is introduced into an allele of the gene of interest, the transcription of the reporter marker sequence is directly controlled by the transcription initiation sequence of the gene of interest.

F. Homozygous Knockout Cell Library, Reporter Cell Library and Homologous Recombination Vector Library

Two or more alleles of a gene in a target cell may be disrupted by a construct exogenous to the target cell. In one embodiment, a trap construct comprising a target cell selection marker sequence may be inserted, for example, via random insertion, into one allele of a gene in the target cell. The target cell is selected and multiplied under selection conditions for the target cell selection marker encoded by the trap construct. A homologous recombination vector, which comprises the trap construct flanked by parts of the genomic sequence of the disrupted gene, can be prepared from the target cell using one of the methods described above. Preferably, the target cell selection marker sequence of the trap construct in the homologous recombination vector is then replaced with a new target cell selection marker sequence that confers a different selectable trait to the target cell. Any other element in the trap construct also can be replaced.

In another embodiment, the genomic sequence of the disrupted gene, or part thereof, may be first determined using PCR, RACE or other methods. A first and/or a second genomic sequence in the disrupted gene, or their homologous sequences, may be selected for constructing a homologous recombination vector in which a new target cell selection marker sequence is flanked by the first and second genomic sequence or their homologous sequences. The new target cell selection marker sequence preferably confers a different selectable trait than that conferred by the trap construct. The first and/or second genomic sequence also may be selected from available genome database, gene expression database, or other sources.

The homologous recombination vector, derived from a target cell in which one allele of a gene has already been disrupted by a trap construct, may be introduced into the cell or its clone. The homologous recombination vector comprises a new target cell selection marker sequence, which preferably confers a different selectable trait than that conferred by the original target cell selection marker sequence in the trap construct. Homologous recombination between the vector and a second allele of the gene can be selected, by virtue of expression of both the selectable traits conferred by the new and the original target cell selection marker. The target cell thus selected has two alleles of the gene disrupted by target cell selection marker sequences, the first allele being disrupted by the original target cell selection marker sequence and the second allele being disrupted by the new target cell selection marker sequence. Other allele(s) of the same gene in the target cell, if exist, also can be disrupted using the same method.

In one embodiment, the new target cell selection marker sequence and the original target cell selection marker sequence may be identical. In such a case, homologous recombination at a second allele may be selected by the expression of a potentially stronger selectable trait conferred by two copies, as compared to only one copy, of the target cell selection marker sequence, provided that the selectable trait conferred by two copies of the target cell selection marker sequence is practically discernable from that conferred by only one copy.

By means of the method described above, various types of mutated cells may be prepared, including homozygous knockout cells. In one embodiment, at least two alleles of a gene in the genome of a target cell are disrupted, a first allele being disrupted by a target cell selection marker sequence and a second allele being disrupted by a reporter marker sequence. To prepare such a target cell, the first allele may be disrupted by a trap construct comprising a target cell selection marker sequence. A homologous recombination vector comprising the target cell selection marker sequence may be prepared from the target cell, using the methods described above. The target cell selection marker sequence in the vector may be then replaced with a cassette sequence comprising a reporter marker sequence and a new target cell selection marker sequence. Homologous recombination between the vector and the second allele may be selected for both the selectable traits conferred by the original and the new target cell selection marker sequence. In another embodiment, the cassette sequence comprises only the reporter sequence. Thus, homologous recombination between the vector and the second allele may be selected for both the traits conferred by the original target cell selection marker sequence and the reporter marker sequence.

In another embodiment, at least two alleles of a gene in a target cell are disrupted, each being disrupted by a reporter marker sequence. The reporter marker sequences at the different alleles of the disrupted gene may be identical or different. Such a target cell may be obtained, for example, if the trap construct that is used to disrupt a first allele of the gene comprises a reporter marker sequence. A homologous recombination vector comprising the trap construct then is prepared, and the original reporter marker sequence in the trap construct is replaced with a new reporter marker sequence. A second allele of the gene can be disrupted by the homologous recombination vector.

Homologous knockout cells with all alleles of a gene disrupted by either target cell selection marker sequences or reporter marker sequences, or both, may be prepared using the above-described methods, as appreciated by one of skill in the art.

In a preferred embodiment, the homologous recombination vector used in the present invention further comprises a negative selection marker, such that the homologous recombination event between the vector and an allele of the gene of interest may be selected using the positive/negative selection methods. The homologous recombination vector in this embodiment comprises (1) a cassette, comprising a trap construct flanked by a first and a second genomic sequence or their homologous sequences, and (2) a negative selection marker sequence which is located either 5′ or 3′ to the cassette, wherein the trap construct comprises a positive selection marker sequence, and wherein the first and second genomic sequence are parts of a gene in a target cell. For the positive/negative selection methods, see U.S. Pat. No. 5,464,764.

Alternatively, a positive selection switch can be used. In this embodiment, a transcription termination sequence, such as polyA, can be placed at the 5′ end of the homologous recombination vector, with a positive selection marker sequence (preferably, a promoter-less marker sequence) downstream from the transcription termination sequence. In this manner, a desired recombination event will cleave the transcription termination sequence, and the downstream positive selection marker sequence will be transcribable (the switch is “on”). On the other hand, if the recombination event is integrated not specifically at an intended site, the transcription termination sequence should not be cleaved upon integration to render the positive selection marker sequence untranscribable; that is, the switch is “off.”

A plurality of target cells, in which each cell has at least one allele of a gene disrupted by a trap construct or a homologous recombination vector or an exogenously introduced construct, constitute a target cell library. In one embodiment, each cell in the library has only one allele of a gene disrupted. In another embodiment, each cell in the library has at least two alleles of a gene disrupted. In yet another embodiment, each cell in the library has all alleles of a gene disrupted. A disrupted gene may be either actively transcribed or silent. The target cell library of the present invention preferably comprises mammalian cells, such as murine or human cells. The cell library also may comprise embryonic stem (ES) cells, such as murine ES cells. A cell library may comprise another cell library.

The cell library of the present invention preferably consists of clones of a single parent cell. A clone of a parent cell may be produced by dividing the parent cell. All subsequent derivations of clones from the parent cell and its clones are said to be genetically identical to the parent cell.

Preferably, the genomes of the different cells present in a given library are essentially identical. For example, they may be derived from a common source or inbred strain, except for the location of the inserted exogenous construct. In a preferred embodiment, the genome of a cell, except for the location of the inserted exogenous construct, in a cell library has at least 95% nucleotide sequence identity, preferably at least 99% nucleotide sequence identity, and most preferably at least 99.9% nucleotide sequence identity, including 100% sequence identity, when compared to the genome of any other cell in the library.

In a preferred embodiment, every cell in a cell library comprises the same trap construct, which disrupts at least one allele of a gene in any given cell in the library.

In another embodiment, a cell library, in which each cell has at least one allele of a gene disrupted, may be prepared using transposable elements. For instance, a transposon comprising either an origin of replication or a host cell selection marker sequence may be constructed, and introduced into the target cells. The genomic sequences adjacent to the transposon may be isolated, sequenced, or used to prepare homologous recombination vectors. These homologous recombination vectors can be used to prepare homozygous knockout cells.

The instant invention also allows for the disruption of a polynucleotide sequence by the random integration of a “transposon-tagged” trapping construct into a genome of a cell by transposase activity. In one embodiment, a trap construct of the instant invention may be modified so as to include, an inverted repeat sequence at its 5′-end and at its 3′-end, such as those recognized by the Tn5 transposase. Consequently, when exposed to a transposase enzyme, such a construct will become randomly integrated into DNA of a target cell and therefore serves as a means to introduce the trap vector containing an origin of replication and/or selection marker into a cell.

In another embodiment, a trap construct of the instant invention may be modified so as to include, an inverted repeat sequence at its 5′-end and at its 3′-end, such as those recognized by the Tn5 transposase. Consequently, when exposed to a transposase enzyme, such a construct can become randomly integrated into purified DNA in vitro obtained from a target organism and preferentially from a target cell. Thus, the transposon/transposase is used to introduce the trap vector into purified genomic DNA. Targeting a genomic DNA with a “transposon-tagged” trapping construct will, therefore, result in the random distribution of the construct throughout the genome. Thus, integration may occur in non-transcribed regions, into exons or introns of transcribed regions of a genome, or downstream of promoters. The DNA containing inserted vector can be recovered with portions of genomic DNA (transposon captured DNA) to generate libraries of DNA, preferably phage-based libraries which can be amplified in suitable host cells.

It may be desirable to screen for recovered DNA (e.g., using gene trap vectors or transposon associated vectors in cells) or transposon captured DNA that have captured promoters from the genomic DNA. In one embodiment, to achieve this, the recovered DNA or transposon captured DNA can be used to transfect or infect target cells. Only the selection marker of the recovered DNA or transposon captured DNA sequences that are downstream of a promoter active in the target cell will result in expression of the selection marker. It may be desirable to block read-through transcription of promoters upstream to the recovered DNA or transposon captured DNA. This can be accomplished by the use of “silencer elements”, preferably placed 5′ to the recovered DNA or transposon capture DNA. Preferred silencer elements are transcription termination sequences and splice donor sequences. In this manner, only those integrated vectors having a trapped promoter within the transposon captured DNA will be transcribed.

In preferred embodiments, this transposon captured DNA can be recovered to determine the identity of the genomic DNA associated with the transposon captured DNA and/or to generate homologous recombination vectors, for example using methods such as those described with the gene trap vectors and produce the libraries, cells and other elements so described.

Accordingly, the instant invention provides a method for integrating a trap construct into a cell genome comprising introducing into a cell, (i) a trap construct of the instant invention and (ii) a transposase enzyme that recognizes inverted repeat sequences engineered into the trap construct, wherein the transposase induces the integration of a part of the construct into the genome. The genome of a cell may or may not be isolated from the cell.

Clones containing transposon-integrated trap constructs can be recovered by any one of a number of standard plasmid rescue methods. Such rescued plasmids can then be identified by sequence analysis and used, with or without modification, as homologous recombination vectors.

A cell library of the present invention may comprise, for example, at least 2 or more cells. A cell library may contain between 5-10, 10-20, 20-30, 30-40, 40-50, 50-100, 100-500 or more than 500 cells, preferably at least about 1,000 cells, more preferably at least about 5,000 cells, highly preferably at least about 10,000 cells, and most preferably at least about 20,000 cells. For example, the presently described cell library may comprise at least about 30,000 cells, at least about 40,000 cells, at least about 50,000 cells, at least about 60,000 cells, at least about 70,000 cells or at least about 80,000 cells, such as 100,000 cells or more.

The cell library may represent, for example, anywhere from 1 to 25 modified or disrupted genes, at least about 25 different genes, or at least about 50 different genes, preferably at least about 100 different genes, more preferably 1,000 different genes, highly preferably 5,000 different genes, and most preferably 10,000 different genes, such as at least 20,000 different genes. For example, the cell library may represent at least about 40,000, or at least about 75,000, different genes. Each of these represented genes corresponds to a cell in the cell library, and at least one allele of the gene is disrupted in the corresponding cell by a trap construct or an exogenously introduced construct, preferentially, more than one allele of the gene is disrupted. In one embodiment, the cell library consists of clones of a single parent cell. The number of disrupted genes in the cell library may be up to the maximum number of genes present in the genome of the parent cell.

A cell library can be essentially a collection of a cells , either maintained in individual liquid stocks or grown as a mixed, single liquid stock. A cell library, therefore, may be a collection of cell cultures each of which represents cells containing an allele disrupted by the inventive methodology. In this regard, a cell library containing alleles disrupted by a construct of the instant invention, also may comprise cell colonies isolated on growth media in a culture dish. For instance, each colony on the culture dish can comprise a disrupted allele that may be the same allele disrupted in other colonies that are stored on the same culture dish.

Alternatively, the cell library may comprise a mixture of cell cultures in one liquid stock solution. In both cases, a cell culture may contain the same or different disrupted allele to another cell culture in the library. In another embodiment, therefore, the disrupted gene in a given cell in a cell library is different from the disrupted gene in any other cell of the library. The cell library of this embodiment may be part of or a subset of another cell library.

Alternatively, a cell library may contain cells each of which contain the same disrupted allele. In this case, the nature of the so-called disruption, such as insertion of a trap construct into the allele, a genetic modification or a nucleotide mutation, may be identical in each of the cells containing the disrupted allele. That is, each cell may contain an allele that has the same mutation or modification. Alternatively, the allele may be disrupted by an assortment of different mutations, modifications or trap locations. If so, the cells of the cell library, while containing the same disrupted allele, may comprise different mutations in that gene allele.

In yet another embodiment, the genome of each cell in a cell library comprises an allele of a gene comprising a construct that is exogenous to the cell. In addition, the allele in a given cell in the library, if without the exogenous construct, encodes a polypeptide that has an amino acid sequence different from that encoded by the allele in any other cell in the library. The cell library of this embodiment may be part of another cell library.

In yet another embodiment, the genome of each cell in a cell library comprises two alleles of a gene, each allele comprising a construct that is exogenous to the cell. In addition, each of the two alleles in a given cell in the library, if without the exogenous construct, encodes a polypeptide that has an amino acid sequence different from that encoded by each of the two alleles in any other cell in the library. The cell library of this embodiment may be part of another cell library.

In one embodiment, a cell library of the present invention may be prepared by introducing a trap construct into a plurality of target cells. These trap constructs, comprising a target cell selection marker sequence, may insert into the genomes of the target cells, disrupting different genes in the genomes. The cells with disrupted genes may be selected for the selectable trait conferred by the target cell selection marker sequence. Preferably, each cell thus selected has only one allele of a gene disrupted by the trap construct. The selected cells or their clones consist of a cell library of this embodiment.

In another embodiment, the other allele or alleles of the disrupted gene in each cell in the library may be disrupted by a homologous recombination vector prepared using one of the methods as described above. The cells thus produced consist of a cell library, in which each cell has at all alleles or at least two alleles of a gene disrupted by either a target cell selection marker sequence or a reporter marker sequence. Each of the alleles may be disrupted by the same or different marker sequences. For example, a cell library may be made, in which each cell has at least two alleles of a gene disrupted, a first allele being disrupted by a target cell selection marker sequence and a second allele being disrupted by a reporter marker sequence. For another example, each cell in a cell library has at least two alleles of a gene disrupted, a first allele being disrupted by a first reporter marker sequence and a second allele being disrupted by a second reporter marker sequence. The first and second reporter marker sequences may be identical or different.

As described above, a polynucleotide comprising part of the disrupted gene in a cell in a cell library may be recovered from the cell, for example, either by reverse transcribing the mRNA derived from the disrupted gene, or by isolating a genomic DNA fragment that comprises part of the disrupted gene. The polynucleotide thus recovered represents the disrupted gene, as well as represents the cell in which the gene is disrupted. Sequencing of the recovered polynucleotide may enable further identification of the disrupted gene. These recovered and sequenced polynucleotides constitute a polynucleotide library, in which each polynucleotide corresponds to a cell in the cell library as well as the gene disrupted in the cell. The scope of this polynucleotide library, and thus the corresponding disrupted genes, preferably encompasses the entire, or nearly entire, set of genes in the cell library. For instance, the polynucleotide library may contain a substantially complete representation of every gene in the cell library. For the purposes of the present invention, the term “substantially complete representation” shall refer to the statistical situation where there is generally at least about an 85-95 percent probability that the genome or transcribed regions of the genomes of the cells used to construct the cell library collectively contain an stably inserted trap construct in at least about 50 percent, preferably at least about 70 percent, more preferably at least 80 percent, highly preferably at least 90 percent, and most preferably at least about 95 percent of the genes present in the cellular genomes or transcribed regions of the genomes, as determined by a standard Poisson distribution, with the assumption that the trap construct inserts randomly.

The polynucleotide library thus prepared can be used to prepare polynucleotide arrays, which are capable of detecting each gene in the cell library. Each polynucleotide of the polynucleotide library also can be used to make a homologous recombination vector, for example, using the methods described above. The homologous recombination vector is directed to the gene part of which is comprised in the corresponding polynucleotide. The homologous recombination vector may comprise a target cell selection marker sequence or a reporter marker sequence. The homologous recombination vectors thus prepared constitute a homologous recombination vector library. The scope of the homologous recombination vector library may contain a substantially complete representation of every gene in the cells of a cell library of the present invention.

In one embodiment, a homologous recombination vector library may be constructed using information within a genome database or a gene expression database. For example, each gene in the genome database or the gene expression database may be identified and a homologous recombination vector directed to the gene, and comprising part of the sequence of the gene, may then be prepared. The homologous recombination vectors so prepared compose a vector library, representing the entire set of the genes, or any subset thereof, in the genome database or the gene expression database. A target cell selection marker sequence or a reporter marker sequence may be included in each homologous recombination vector.

In one embodiment, mouse ES cells, such as early passage mouse ES cells, are used to construct a cell library of the present invention. The cell library thus made becomes a genetic tool for the comprehensive study of the mouse genome. Since ES cells can be injected back into a blastocyst and incorporated into normal development and ultimately the germ line, the mutated ES cells in the library effectively represent collection of mutant transgenic mouse strains. The resulting phenotypes of the mutant transgenic mouse strains, and therefore, the function of the disrupted genes, may be rapidly identified and characterized. The resulting transgenic mice may also be bred with other mouse strains and back crossed to produce congenic or recombinant congenic animals that allow for the evaluation of the trap mutation in different genetic backgrounds. A representative listing various strains and genetic manipulations that can be used to practice the above aspects of the present invention (including the ES cell libraries) can be found in Genetic Variants and Strains of the Laboratory Mouse, 3rd Ed., Vols. 1 and 2, Oxford University Press, New York, 1996.

A similar methodology can be used to construct virtually any non-human transgenic or knockout animal. These nonhuman transgenic or knockout animals include pigs, rats, rabbits, cattle, goats, non-human primates such as chimpanzee, and other animal species, particularly mammalian species.

Any trap construct or homologous recombination vector described in the present invention can be employed to make a cell, a cell library, or a transgenic or knockout animal, as described above.

By the same token, the inventive method also may be used for the purposes of producing one modified cell, one polynucleotide or one type of vector. That is, the invention may applied to single use and not solely for the generation of a library, per se. For instance, there is no presumption that the creation of a cell culture containing a modified or disrupted gene or allele by the inventive method, must comprise part of a cell library. Similarly, an isolated polynucleotide or vector of the instant invention is not necessarily a member of a polynucleotide or vector library.

G. Down-regulation

In unmodified cells, single copy knockout cells or multiple copy knockout cells that still express the targeted gene product, methods and reagents can be used to down-regulate the expression of the targeted gene product.

Examples of such down-regulation include, but are not limited to, the use of (i) antisense sequences, (ii) double-stranded RNA (dsRNA), (iii) catalytic RNA (ribozyme), etc.

Such down-regulation systems generally target a specific nucleotide sequence in the genomic DNA or mRNA transcripts. For example, according to the instant invention, double-stranded RNA also may be used to disrupt the expression of a gene or polynucleotide of interest. A dsRNA molecule can be introduced into a cell that targets and induces degradation of an mRNA that is derived from a gene or polynucleotide of interest. The exact mechanism of how the dsRNA targets the mRNA is not essential to the operation of the invention, other than the dsRNA shares sequence homology with the mRNA transcript. The mechanism could be a direct interaction with the target gene, an interaction with the resulting mRNA transcript, an interaction with the resulting protein product or another mechanism.

Again, while the exact mechanism is not essential to the invention, it is believed the association of the dsRNA to the target gene is defined by the homology between the dsRNA and the actual and/or predicted mRNA transcript. It is believed that this association will affect the ability of the dsRNA to disrupt the target gene.

As an example, different dsRNA sequences (or other down-regulation agents) can be synthesized and screened for effectiveness to optimize a down-regulation dsRNA target sequence and associated dsRNA agent.

Thus, designing a dsRNA molecule which is complementary to a specific mRNA sequence of a trapped gene is a straightforward procedure. In the case of a polynucleotide obtained using the 5′ trap, for example, a sequence could be designed that is upstream of the vector sequence. Similarly, the sequence downstream of the vector sequence can be used in the design of a dsRNA molecule, if the trapped gene is obtained using a 3′ trap of the instant invention.

However, the sequence from which a dsRNA molecule may be designed is not limited to sequences obtained via “trapped” genes. A dsRNA molecule for use in the present invention may be designed from database-submitted entries, via data obtained from techniques such as RACE, or via other methods that can determine the identity of the trapped gene, such as through the use of polynucleotide arrays. For instance, one may validate the sequence integrity of identified dsRNA molecules, by applying the dsRNA molecule to gene arrays and identifying which gene(s) or gene fragment(s) they hybridize to. The individual RNA strands that make up the dsRNA molecule can be made recombinantly or synthesized chemically. The resultant dsRNA may be introduced into reporter cells of the instant invention by one of any standard techniques such as transfection, lipofection and electroporation, or viral delivery systems.

In any event, prior to using a dsRNA molecule to modulate the expression of an endogenous gene in vivo, it may be necessary to identify the effectiveness of a dsRNA molecule in causing the degradation of an mRNA transcript, by evaluating its effect on a chimeric reporter-gene mRNA transcript. In this regard, a reporter gene is fused to a gene of interest, such that a single reporter-gene fusion product is translated from an intact mRNA transcript. However, degradation of any part of the mRNA transcript may preempt translation of either protein. Thus, the measurable activity of the reporter can be an indicator of the stability of the mRNA transcript. The effectiveness of a dsRNA molecule in bringing about the degradation of an mRNA transcript, and thus the down-regulation of a gene, can be tested by following the activity of a reporter marker. The reporter marker may encode a fluorescent protein.

Accordingly, the instant invention provides a method for evaluating the effectiveness of a dsRNA molecule prior to its use in modulating an endogenous gene. In preferred embodiments, the procedure entails creating a construct comprising, in the 5′-3′ order and operably linked to one another, a promoter, gene of interest, IRES sequence and a reporter polynucleotide or the same functional elements without the use of an IRES. The position of the gene of interest and the reporter polynucleotide may be interchanged. In any event, the resultant mRNA transcript comprising the gene of interest and the reporter polynucleotide, would be susceptible to nuclease activity induced by the action of a dsRNA molecule designed to be complementary to some part of the resultant mRNA transcript. The dsRNA molecule could be complementary to some part of the gene of interest, such that it induces degradation of the gene of interest portion of the resultant mRNA transcript. Depending on the activity of the reporter molecule, one can follow the stability of the mRNA transcript and, consequently, record the amount of protein expressed.

This system allows the skilled artisan to expose a variety of dsRNA molecules with different sequences to a cell expressing the described construct. As a result, the skilled artisan may identify dsRNA sequences that are particularly good at inducing degradation of the mRNA transcript as opposed to other dsRNA sequences which do not. It follows, therefore, that the skilled artisan can determine the effectiveness of a dsRNA in modulating gene activity by following the activity of the reporter protein.

Thus, by designing dsRNAs to different regions of a trapped gene, one may select a sequence that for some reason is particularly efficient in inducing nuclease activity which degrades the mRNA. Accordingly, the inventive method provides an efficient method of determining which dsRNA molecules best modulates a specific, endogenous gene. The term “modulates” means the partial or complete down-regulation of a gene.

In this regard, a dsRNA molecule of the present invention, identified by the above-described method as capable of modulating a gene in vitro, may be administered to an individual to determine whether it has effect in vivo. In accordance with the invention, therefore, the dsRNA may be prepared in a suitable formulation for in vivo administration. The subject may be any animal, such as a mouse or a rat, or the subject may be a human. A dsRNA may function in vivo as a drug, in the sense that the dsRNA may reduce expression of a gene that either is not expressed normally, is expressed during a specific stage of cell development or age, or is over-expressed due to some genetic disorder or abnormality. In this regard, administering an amount of a dsRNA of the instant invention that is effective in modulating the expression pattern of a specific gene represents a therapeutic application.

To facilitate the use of an inventive dsRNA molecule as a therapeutic agent, the dsRNA molecule may be protected against nucleic acid degradation by any one of a number of known techniques. For instance, a formulation of dsRNA may be encapsulated within a liposome, prior to administration. A formulation of nucleic acid and polyethylene glycol, for instance, may also increase the half-life of the nucleic acid in vivo, as could any known slow-release nucleic acid formulation. Other methods may be used to protect and enhance the bioavailability of a nucleic acid. For example, a thiol group may be incorporated into a polynucleotide, such as into an RNA or DNA molecule, by replacing the phosphorous group of the nucleotide. When so incorporated into the “backbone” of a nucleic acid, a thiol can prevent cleavage of the DNA at that site and, thus, improve the stability of the nucleic acid molecule.

Accordingly, a phosphorothioate-modified oligonucleotide is one type of nucleic acid derivative that may be administered to an individual. Other modified oligonucleotide backbones include, for example, those described in U.S. Pat. No. 6,323,029, which is incorporated herein by reference. The '029 patent describes modifications for an oligonucleotide that is used in antisense suppression of gene expression. For instance, a nucleic acid molecule backbone may be modified so as to contain phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

These, and other approaches to protecting or stabilizing a nucleic acid are well known. For instance, see “Synthesis of Modified Oligonucleotides,” Ortiago & Rösch, Interactiva homepage at http://www.interactiva.de/knowledge/nucleicchem/modifiedoligos.html of that company's website, checked on Feb. 26, 2002, and U.S. Pat. No. 5,965,721 which are incorporated herein by reference. The latter also describes nucleic acid analogues that have improved nuclease resistance and improved cellular uptake.

Yet another method of modifying a nucleic acid of the instant invention involves the production of a “locked nucleic acid” (LNA). Typically, an LNA is characterized by a methylene linker that restricts the normal conformational freedom of the furanose ring in a nucleoside. This linker typically connects together the 2′-O and 4′-C of a furanose ring and prevents or reduces its normal degree of conformational freedom. These particular LNA oligomers obey Watson-Crick base pairing rules and will hybridize to complementary oligonucleotides. What is more, LNA/DNA and LNA/RNA duplexes have increased thermal stability and half-life, as well as enhanced affinity and specificity when compared to duplexes formed by DNA or RNA. In general, the thermal stability of a LNA/DNA duplex is increased 3° C. to 8° C. per modified base in the oligonucleotide. See, for instance, Wahlestedt et al., Proc. Natl. Acad. Sci., 97 (10), 5633-5638, 2000 and U.S. Pat. No. 6,303,315.

LNA oligonucleotides can be synthesized using standard phosphoramidite chemistry using DNA-synthesizers and can be mixed with other standard DNA and RNA oligonucleotides to produce a mixed preparation of modified and unmodified nucleic acid molecules. It is also possible to synthesize LNA with standard 3′ and/or 5′-modifiers, such as with aminolinker, biotin, Cy3, Cy5, or fluorescent markers for example.

Furthermore, fully modified LNA oligonucleotides are resistant towards most nucleases, enter cells efficiently and are not toxic to the animals in which they are administered. See Wahlestedt et al., supra. Thus, these features make LNA a useful tool in biological research, DNA diagnostics and in the development of therapeutic drugs.

In this respect, the bioavailability of a nucleic acid treatment in vivo may also be improved by modifying the nucleic acid according to the instant invention. For instance, a dsRNA may be modified and formulated so that it has an increased half-life and/or is retained in plasma for longer periods of time than non-modified dsRNAs. Thus, modifying a nucleic acid, such as a dsRNA molecule of the instant invention, may increase the effectiveness of the dsRNA in vivo and/or its bioavailability.

Accordingly, after determining the effectiveness of a dsRNA molecule in modulating the expression of a gene in vitro, pursuant to the invention, the molecule may be modified so as to improve its resistance to degradation and administered to an individual by one of the methods described above. Hence, the expression of a gene may be partially or completely down-regulated in an individual treated with a modified dsRNA, thereby altering a phenotype associated with that individual. The phenotype may be a normal one or may be associated with a disease or some other abnormality.

The inventive method may be used to indirectly upregulate the expression of a target gene whose expression is inhibited, or reduced by a second gene, by designing a dsRNA molecule to target and bind to the second gene's mRNA transcript, thereby causing its degradation. The effect of the second gene upon the target gene may be normal, or it may be a consequence of an abnormal imbalance induced by a disease state.

The inventive method envisions the creation of dsRNA libraries whereby each dsRNA is capable of reducing either completely, or to some extent, the level of expression of a specific gene. The inventive method also envisions the creation of cell libraries wherein each cell in the library contains a modulated target gene that is different to the target genes modulated in other cells of the library. As used herein, a “dsRNA-cell library” represents a collection of cells, colonies or cultures that contains a dsRNA-modulated gene. A dsRNA cell library may contain numbers of cells as described above or anywhere from 2 to 10 cells, colonies or cell cultures representing an assortment of different or the same dsRNA-modulated genes. Thus, a dsRNA cell library may represent, for example, anywhere from 2 to 25 modified or disrupted genes, at least about 25 different genes, or at least about 50 different genes, preferably at least about 100 different genes, more preferably 1,000 different genes, highly preferably 5,000 different genes, and most preferably 10,000 different genes, such as at least 20,000 different genes. For example, the cell library may represent at least about 40,000, or at least about 75,000, different genes.

Also provided is an alternative way to use dsRNA to modify gene expression. Pursuant to the present invention, a cell that has had an allele inactivated or disrupted, due to a single homologous recombination event, can be exposed to a dsRNA molecule that is associated with the other copy, or allele, of that knocked out gene. Consequently, the level of expression of the remaining allele(s) will be modified by the introduced dsRNA molecule.

The expression of an exogenous gene or polynucleotide may also be modulated by dsRNA techniques. For instance, a unique polynucleotide sequence may be incorporated into a vector of the instant invention, which, when transcribed into an mRNA transcript, can be targeted by a complementary dsRNA molecule. In such fashion, the skilled artisan is able to readily modulate the expression of a polynucleotide introduced into a host or target cell.

On a larger scale, a transgenic animal comprising cells transformed with an exogenous gene and construct may be subjected to dsRNA molecules, such as in feed, liquids, injection or other means known to those in the art, to inactivate or down-regulate expression of the exogenous DNA.

Other methods such as those described with dsRNA techniques can be used with antisense sequences, ribozymes etc.

H. Applications of the Present Invention

The following demonstration of the utilities of the present invention is given by way of illustration only. Other uses of the present invention are virtually unlimited. For instance, essentially any previous known uses for trapping constructs, homologous recombination vectors, microarrays, cells, cell libraries, cDNA libraries, and transgenic or knockout animals may be addressed using the presently described trap constructs, homologous recombination vectors, microarrays, cells, cell libraries, polynucleotide libraries, and transgenic or knockout animals.

Transgenic animals and cells prepared using the present invention are useful for the study of basic biological processes as well as diseases, including, but not limited to, aging, cancer, autoimmune disease, immune disorders, alopecia, glandular disorders, inflammatory disorders, ataxia telangiectasia, diabetes, arthritis, high blood pressure, atherosclerosis, cardiovascular disease, pulmonary disease, degenerative diseases of the neural or skeletal systems, Alzheimer's disease, Parkinson's disease, asthma, developmental disorders or abnormalities, infertility, epithelial ulcerations, and viral and microbial pathogenesis and infectious disease. See, for example, Principles and Practice of Infectious Disease, 3rd Ed., Churchill Livingstone Inc., New York, 1990.

In addition, the presently described trap constructs, methods, libraries, cells, and animals are equally well suited for identifying the molecular basis for genetically determined advantages such as prolonged life-span, low cholesterol, low blood pressure, resistance to cancer, low incidence of diabetes, lack of obesity, or the attenuation of, or the prevention of, all inflammatory disorders, including, but not limited to coronary artery disease, multiple sclerosis, rheumatoid arthritis, systemic lupus erythematosus, and inflammatory bowel disease.

The cell libraries of the present invention can be exposed to many different kinds of assays to evaluate, for example, response to growth factors and cytokines, production of biochemical markers of a disease (such as an enzyme), or biological capabilities (such as adhesion, invasiveness, or growth characteristics). The cell libraries may comprise 2 or more, 5-10, 10-20, 20-30, 30-40, 40-50, 50-100, 100-500 or more than 500 cells. Each cell in such a library may comprise a disrupted allele that is different to a disrupted gene allele in another cell of the library. Alternatively, the library may contain multiple cells each containing the same disrupted allele.

The polynucleotide arrays of the present invention may be used to identify over-expressed or under-expressed genes in diseased cells, such as tumor cells, e.g. colon cancer cells. The presently described cells, in which at least one allele of a gene is disrupted, including homozygous knockout cells, can be used to identify the phenotypes or effects associated with the disruption or inactivation of the genes. These phenotypes or effects include, but are not limited to, anchorage independent growth, production of angiogenic factor or metastasis, tumorigenesis in animals, and responsiveness to chemotherapeutic agents.

The cell library comprising homozygous knockout cells can be used to identify genes that are essential for biological attributes of a diseased cell. For example, the homozygous knockout cell library derived from a diseased cell may be employed to identify the gene(s), inactivation of which ablates the diseased phenotype. The homozygous knockout cells also can be used to identify the function of a gene by monitoring the biochemical or physiological effect of the inactivation of the gene.

In one embodiment, the therapeutic or diagnostic utility of an over-expressed gene in a diseased cell may be identified using the present invention. For example, genes that are over-expressed in diseased cells, such as tumor cells including Kras transformed colon cancer cells, can be identified using a polynucleotide array of the present invention.

Homologous recombination vectors directed to the identified, over-expressed genes may also be prepared using one of the methods as described above or other methods as appreciated in the art. These homologous recombination vectors can be introduced into the diseased cells to inactivate any one of these over-expressed genes, for example, by disrupting any or all alleles of the gene in the genome of a diseased cell. Such inactivation may be facilitated by using a target cell library, which comprises an easily identifiable cell in which at least one allele of any one of the identified, over-expressed genes has already been disrupted.

In another embodiment, the inactivation of an identified, over-expressed gene may be achieved by directly choosing, from a homozygous knockout cell library, a cell in which all alleles of the gene have already been disrupted. The biological or biochemical effects of the inactivation of an over-expressed gene may be evaluated using different biological or biochemical assays, as appreciated the art. For example, these effects may relate to anchorage independent growth, production of angiogenic factors or metastasis, growth in low nutrients, growth factor independent growth, autocrine growth, alternation of signal transduction pathways (for example, Ras, p53, growth factor receptor signaling, and lipid metabolism), tumorigenesis in animals, or responsiveness of the cell to chemotherapeutic agents or radiation. The therapeutic utility of the inactivated gene therefore can be determined.

In one embodiment, the homologous recombination vector also comprises a reporter marker sequence. A drug or compound library may be applied to the disease cell, in which the reporter marker sequence is inserted into one allele of an expressed gene, to screen for candidates that may inhibit or reduce the expression of the reporter marker sequence, and therefore, the expression of the expressed disease gene.

In another embodiment, genes involved in a diseased phenotype of a diseased cell, but not over-expressed in the diseased cell, can be identified using the present invention. For instance, a trap construct may be introduced into the diseased cells to disrupt a large number of the genes. Homologous recombination vectors directed to the disrupted genes may be prepared, using one of the methods as described above. The homologous recombination vectors are used to inactivate other alleles of the disrupted genes in the cells. Some of the cells thus made may show a lesser degree of the diseased phenotype, suggesting that the genes inactivated in these cells may be responsible for the development of the diseased phenotype. The sequence of these disease genes may be determined, for example, using the polynucleotide array or the polynucleotide library of the present invention. In addition, a reporter marker sequence can be introduced to one allele of the diseased genes, for example, via homologous recombination vectors, such that drugs or compounds affecting the expression of the disease genes may be identified.

In yet another embodiment, genes involved in a diseased phenotype of a diseased cell, but either under-expressed or not expressed in the diseased cell, may be identified using the present invention. For instance, the non-expressed or under-expressed genes in the diseased cells may be first identified using a polynucleotide array of the present invention, when compared to the gene expression in normal cells. Homologous recombination vectors directed to these under-expressed or not expressed genes may be prepared, and used to inactivate any one of these genes in cells that do not originally have the diseased phenotype. The cells thus modified are screened for the diseased phenotype in order to identify the gene or genes that may be involved in the development of the phenotype. A homologous recombination vector with a reporter marker sequence also may be used, to introduce the reporter marker sequence into one allele of the under-expressed or not expressed genes. Drugs or compounds that induce or increase the expression of these genes may be identified. In a particular embodiment, an available homozygous knockout library may be used to screen for the cells showing the diseased phenotype, and the responsible genes then may be identified using a polynucleotide library representing the genes inactivated in the knockout cell library.

In one embodiment, the cell in which one, two or all alleles of a gene are disrupted by a trap construct or a homologous recombination vector, may be used to screen for compounds that regulate the expression of the disrupted gene. For example, the trap construct or the homologous recombination vector, lacking a transcriptional initiation sequence functional in the cell, may comprise a reporter marker sequence. The cell may be subject to a compound or drug library to screen for the compounds or drugs that affect the expression of the reporter marker sequence, for example, by comparing the expression of the reporter marker before and after contacting a particular compound or drug.

In another embodiment, the trap construct of the present invention can be used to identify compounds capable of inducing expression of a silent gene in a target cell. For instance, a trap construct lacking a transcriptional initiation sequence functional in a target cell may be incorporated into the genome of the target cell. The trap construct comprises a positive and a negative target cell selection marker sequence. The two marker sequences may be expressed as a fusion protein, such as bsdS:codA::upp. The target cell is first selected against the negative marker, such as against codA::upp using 5-FC, so that the target cell in which the trap construct is inserted into an actively transcribed genomic sequence is selected out. If the trap construct is inserted into a non-actively transcribed genomic sequence, the target cell may survive the negative selection. Compounds or drugs that are capable of inducing transcription of the non-actively transcribed genomic sequence can therefore be identified by selection for the positive marker of the target cell.

In one embodiment, the homozygous knockout cell of the present invention may be used to determine the effect of inhibition of a potential gene target on transcription of other genes. For instance, RNA expressions in a presently described homozygous knockout cell can be compared to those in control cells. Expression patterns of genes that are affected by the gene knockout in the homozygous cell, can readily be identified and may include therapeutically related genes.

In another embodiment, the present invention can be used to determine the specificity of drug candidates on a chosen target gene. Usually, the more specific the drug candidate is for the desired target gene, the less likely there will be non-target associated toxicity in humans. Because gene inactivation in a representative homozygous knockout cell is specific for the target gene, effects of such inactivation on, for example, other genes' expression, can be used as a “gold standard” to compare to the effects (and “side effects”) of drug candidates on the inhibition of the same target gene. In so doing, it is possible to determine the specificity of drug candidates upon the target gene.

In yet another embodiment, the present invention can be used to identify genes differentially regulated in diseased cells or in response to disease associated stimuli. Stimuli include but are not limited to the activity of a growth factor, a cytokine, or an oncogene. For instance, a promote construct comprising a reporter marker sequence, or a homologous recombination vector comprising a reporter marker sequence, may be introduced into the genomes of diseased cells. The construct or vector may also include a target cell selection marker sequence to allow selecting the modified diseased cells in which at least one allele of an transcriptionally active gene is disrupted by the construct or vector. The diseased cells may be oncogene (e.g. Ras) transformed cells, and the expression of the oncogene in the cells may be regulated, for example, using a suitable promoter. Thus, expression of the oncogene in the cells may be turned on or off, as desired. In the cells with the oncogene in an “off” state, the reporter marker expressions can be compared to the reporter marker expressions in the cells where the oncogene is on. Consequently, the genes regulated by the oncogene may be identified. By the same token, the oncogene in this embodiment may be replaced with another gene, expression or over-expression of which produces a diseased phenotype in cells. Illustrative of such genes are p53 and toxic genes.

In yet another embodiment, the functions of genes from viruses or other pathogens that affect the expression of genes in cells, such as mammalian cells, can be determined using the present invention. Chemicals that modulate these genes also can be identified using the methods of the present invention. Many transforming viruses, after infecting a target cell, have the effect of up-regulating genes involved in cell proliferation, which allows the virus-infected cells to produce additional viruses, which can infect additional cells. These transforming viruses can act by stimulating a receptor from the target cell. One example of the mechanism is the Friend Erythroleukemia virus. This virus uses the erythropoietin receptor for entry into the cells. When the virus is bound to the receptor, a pathway is activated that causes an over-proliferation of red blood cells. If the activation of the erythropoietin receptor is inhibited, a decrease in the accumulation of red blood cells would result which can prevent or reduce the severity of the leukemia. The development of an assay that reports the activation of mammalian target genes allows the identification of modulators of other viral or pathogenic dependent pathways. These modulators can be used as therapeutic agents.

A general procedure for establishing this assay uses the virus or an isolated viral protein as the stimulus for modulating a pathway. First, a target cell library is made using a cell line that can be infected by the virus or activated by the viral protein. Each cell in the library has at least one allele of a gene, preferably two alleles of the gene, more preferably all alleles of the gene, disrupted by a trap construct. The trap construct comprises a reporter marker sequence. The construct preferably is a promoter or an exon trap construct which does not contain a transcriptional initiation sequence functional in the target cell. The virus or an isolated viral protein is added to these cells, and clones that respond specifically to the viral infection, for example, by the expression of the reporter marker are isolated. Chemicals that inhibit this effect also can be screened and identified.

This approach can be applied to any cellular pathogen that has an effect on target cells, such as cytotoxicity, cell proliferation, inflammation or other responses. These cellular pathogen include viruses, such as retroviruses, adenovirus, papiltomavirus, herpesviruses, cytomegalovirus, adeno-associated viruses and hepatitis viruses, viral proteins, or any other pathogen, such as parasites, bacteria and viroids. In addition, two or more viral components can be added to identify coviral pathogenesis components. This is a particularly valuable tool for identifying pathways modulated by two or more viruses concurrently, or over time as in slow activating viral conditions. Suitable cellular pathogens also include oncogenes or proto-oncogenes found in uninfected genomes, or gene products thereof.

In another embodiment, the present invention also provides for a method of identifying proteins or chemicals that directly or indirectly modulate a gene in a target cell. Generally, the method comprises (A) inserting a trap construct or homologous recombination vector of the present invention into one allele of the gene, wherein the trap construct or the homologous recombination vector comprises a reporter marker or a target cell selection marker sequence, or both; (B) contacting the cell with a concentration of a modulator; and (C) placing the cell under conditions for selection of the target cell selection marker encoded the trap construct or monitoring the expression of the reporter marker sequence. The trap construct or the homologous recombination vector preferably is or derived from a promoter or an exon trap construct which does not contain a transcriptional initiation sequence functional in the target cell. The effect on the expression of the target cell selection marker or the report marker before and after contacting with the modulator, as well as the identity of the gene, can be determined.

When a trap construct or a homologous recombination vector comprises a target cell selection marker sequence or a reporter marker sequence and is inserted into an allele of a gene in the genome of a target cell, such that the selection or reporter marker sequence are expressed under a variety of circumstances, then the target cell can be used for drug discovery and functional genomics. The trap construct or the homologous recombination vector preferably is, or is derived from, a promoter trap or an exon trap construct that does not contain a transcriptional initiation sequence functional in the target cell. The target cell that reports the modulation of the expression of the selection marker or the reporter marker sequence in response to a variety of stimuli, such as hormones and other physiological signals, may be identified. Thus, the gene disrupted in the target cell is involved in responding to the stimuli. These stimuli may relate to a variety of known or unknown pathways that are modulated by known or unknown modulators. Chemicals that modulate the target cell's response to the stimuli also can be identified.

In another embodiment, the invention provides for a method of identifying developmentally or tissue specific expressed genes. Trap constructs comprising suitable selection marker sequences can be inserted, for example randomly, into the genome of any precursor cell such as an embryonic or hematopoietic stem cell to create a library of clones. The trap construct preferably is a promoter or an exon trap construct which does not contain a transcriptional initiation sequence functional in the target cell. The library of clones can then be stimulated or allowed to differentiate. Induction or repression of the expression of the selection marker encoded by the trap constructs are determined.

Human disease genes are often identified and found to show little or no sequence homology to functionally characterized genes. Such genes are often of unknown function and thus encode an “orphan protein.” Usually such orphan proteins share less than 25% amino acid sequence homology with other known proteins or are not considered part of a gene family. With such molecules there is usually no therapeutic starting point. In another embodiment, the invention provides for a method to identify modulators of orphan proteins or genes that are directly or indirectly modulated by an orphan protein. By using the cell and polynucleotide libraries described herein, one can extract functional information about these orphan genes.

In one embodiment, orphan proteins can be expressed, and preferably over-expressed, in a cell library, in which each cell has at least one allele of a gene is disrupted by a trap construct or a homologous recombination vector of the present invention. The trap construct or the homologous recombination vector comprises a suitable marker sequence. The genes that are regulated by the orphan proteins may be identified by monitoring the orphan proteins' effect on the expression of the marker sequences. Insights gained using this method can lead to identification of valid therapeutic targets for diseases associated with orphan proteins.

What is claimed is:

1. An array of clones, comprising multiple groups of vessels, of which at least two of said vessels each contain a clone, wherein each clone (i) contains an exogenous segment within a gene of its genome, such that said gene is disrupted, and (ii) is arranged in said array in predetermined fashion.

2. An array of clones, comprising multiple groups of vessels, of which at least two of said vessels each contain a clone, wherein each clone contains an exogenous segment within each allele of a gene of its genome, such that all alleles of said gene are disrupted.

3. An array of clones, comprising multiple groups of vessels, of which at least two of said vessels each contain a clone, wherein each clone contains (i) an first exogenous segment within a first gene of its genome, such that said first gene is disrupted, and (ii) a second exogenous segment within a second gene of its genome, such that said second gene is disrupted.

4. A construct comprising functional elements that, when transcribed, produce a eukaryote mRNA transcript that contains a component selected from the group consisting of an origin of replication and a host cell selection marker.

5. A construct comprising (A) a splice acceptor site, (B) a cassette sequence selected from the group consisting of (i) a transcriptional termination sequence and (ii) a splice donor site; (iii) a cell selection marker sequence; and (iv) an origin of replication, wherein said origin of replication and said cell selection marker sequence are located downstream to the 5′-end of said splice acceptor site and upstream to the 3′-end of said cassette sequence, and wherein said origin of replication is exogenous to said splice acceptor site or said cassette sequence.

6. A construct comprising (i) a transcriptional initiation sequence; (ii) a splice donor site; (iii) a cell selection marker sequence; and (iv) an origin of replication, wherein said origin of replication and said marker sequence are located downstream to the 5′-end of said transcriptional initiation sequence and upstream to the 3′-end of said splice donor site, and wherein said origin of replication is exogenous to said transcriptional initiation sequence or said splice donor site.

7. A library of constructs, comprising more than about 10 of said constructs, wherein each construct of said library produces, upon transcription, an mRNA transcript containing an exogenous origin of replication or a host cell selection marker, and wherein at least some of the mRNA transcripts produced by said constructs represent different gene sequences.

8. A library of constructs, comprising from about 10 to about 50 of said constructs, wherein each construct of said library produces, upon transcription, an mRNA transcript containing an exogenous origin of replication or a host cell selection marker, and wherein each of the mRNA transcripts produced by said constructs represent a region of a cell genome that does not encode a polypeptide.

9. A homologous recombination vector, comprising: (i) a splice acceptor sequence or an IRES sequence; (ii) an origin of replication or a host cell selection marker; (iii) a means for facilitating termination and polyadenylation of an endogenous polynucleotide; (iv) a first genomic fragment recovered from a cell; and (v) a second genomic fragment recovered from a cell, wherein (ii) is downstream of (i) and upstream of (iii), and wherein said first genomic fragment is upstream of (i) and said second genomic fragment is downstream of (iii).

10. A linear construct comprising, in the 5′ to 3′ order, (i) a first nucleotide sequence that is homologous to a first genomic sequence of a cell, (ii) a construct comprising an origin of replication that is exogenous to said cell, and (iii) a second nucleotide sequence that is homologous to a second genomic sequence of said cell.

11. A positive switch homologous recombination vector, comprising the elements: (i) a splice acceptor sequence or an IRES sequence; (ii) a first termination sequence; (iii) a positive selection marker; (iv) a first genomic fragment recovered from a cell; and (v) a second genomic fragment recovered from a cell, wherein, said elements are arranged such that (ii) is upstream of (i), (iii) and (iv) and (v) is downstream of (i), (iii) and (iv).

12. A cell, comprising: (i) an allele of a first gene into which an exogenous polynucleotide has been integrated, wherein said exogenous polynucleotide contains an origin of replication or a selectable marker upstream of a means for facilitating termination and polyadenylation of an endogenous polynucleotide and downstream from a transcription initiation sequence.

13. A cell comprising: (i) a first allele of a first gene into which an exogenous polynucleotide has been integrated by a method other than homologous recombination, and (ii) a second allele of said first gene into which a homologous recombination event has occurred.

14. A multiple-gene disrupted cell, comprising: (i) all alleles of a first gene contain an integrated construct or a portion thereof; and (ii) all alleles of a second gene contain an integrated construct or a portion thereof, wherein said construct comprises an exogenous origin of replication or a host cell selection marker.

15. A library of single-allele disrupted cells, comprising at least two cells each of which comprise an allele of a first gene into which an exogenous polynucleotide has been integrated, wherein said exogenous polynucleotide contains an origin of replication or a host cell selection marker.

16. A library of two-allele disrupted cells that comprises at least two cells, each of said cells comprising (i) a first allele of a gene, into which an exogenous polynucleotide has been integrated by a method other than homologous recombination, and (ii) a second allele of said gene, into which a homologous recombination event has occurred.

17. A library of multiple-gene disrupted cells that comprises at least two cells, each of said cells comprising (i) all alleles of a first gene, containing an integrated construct or a portion thereof, and (ii) all alleles of a second gene, containing an integrated construct or a portion thereof, wherein said construct comprises an exogenous origin of replication or a host cell selection marker.

18. A collection of at least two cells, each comprising an allele of a first gene into which an exogenous polynucleotide has been integrated, wherein said exogenous polynucleotide contains an origin of replication or a selectable marker that is (A) upstream of a means for facilitating termination and polyadenylation of an endogenous polynucleotide and (B) downstream from a transcription initiation sequence, wherein each cell comprises a different gene into which an exogenous polynucleotide is integrated.

19. A method for recovering at least a portion of a gene allele of a cell genome, comprising: (i) providing a cell in which at least one allele of a gene in the genome of said cell contains a construct, or a portion thereof, within any part of its nucleotide sequence, wherein said construct, or a portion thereof, comprises an origin of replication or a host cell selection marker; (ii) recovering a nucleic acid molecule containing said construct, or a portion thereof, that comprises (i) said origin of replication or said host cell selection marker and (ii) nucleic acid derived from said allele; and (iii) isolating said nucleic acid molecule, wherein said nucleic acid derived from said allele flanks either the 5′-end, the 3-′end or both ends of said construct, or a portion thereof.

20. A method for making a homologous recombination vector comprising: (i) integrating a construct into the genome of a cell; (ii) recovering a polynucleotide comprising at least a portion of said construct that is flanked at either its 5′-end or its 3′-end, or at both of its ends by a genomic fragment of said genome; and (iii) isolating said polynucleotide to form a homologous recombination vector.

21. A method of making a homologous recombination vector, comprising: (i) preparing, in vitro, a purified genomic preparation of DNA from a cell; (ii) integrating a construct into said genomic preparation; (iii) recovering a chimeric polynucleotide comprising a portion of said construct with at least one flanking genomic fragment joined to either the 5′-end or 3′-end, or both ends, of said portion of said construct; and (iv) isolating said chimeric polynucleotide to form a homologous recombination vector.

22. A method for determining the function of a gene, comprising: (i) providing a first cell containing at least two disrupted alleles of a gene, and (ii) comparing biological traits of said first cell to those of a second cell in which no alleles of said gene are disrupted, wherein either:
(A) an allele of said first cell contains an exogenous polynucleotide integrated into it by a method other than homologous recombination and at least one other allele contains an exogenous segment integrated by the method of homologous recombination; or
(B) each allele of said first cell is disrupted by a homologous recombination vector.

23. A method for selecting a compound that regulates the expression of a reporter marker integrated into at least one allele of a gene in a cell, comprising (i) contacting a compound with at least one cell of a cell library and (ii) comparing fluorescent light intensity of a reporter marker sequence integrated into said cell before and after contacting said compound, wherein said cell comprises an exogenous segment integrated into its genome and wherein said exogenous segment comprises an origin of replication or a host cell selection marker.

24. A method for determining the effectiveness of a double-stranded RNA molecule, comprising: (i) introducing a construct that comprises (a) a promoter, (b) a polynucleotide of interest, (c) an IRES sequence and (d) a reporter marker into a cell that has one allele disrupted by an exogenous polynucleotide; (ii) determining the activity or expression level of said reporter marker; then (iii) introducing into said cell a double-stranded RNA molecule designed to a portion of said polynucleotide of interest; and (iv) determining the activity or presence of said reporter marker.

25. A messenger RNA transcript isolated from a cell expressing a construct of claim 4, wherein said mRNA transcript comprises a first portion comprised of a polypeptide-encoding region of said cell genome and a second portion comprised of an mRNA transcript of said construct.

26. A construct according to any one of claim 4, 5, 6, 9, 10 or 11, further comprising at least one inverted repeat sequence that is targeted by a transposase enzyme.

27. A method for integrating a trap construct into a cell genome, comprising introducing into a cell (i) a trap construct of claim 26 and (ii) a transposase enzyme that recognizes inverted repeat sequences in said trap construct, wherein said transposase induces the integration of a part of said construct into said genome.
